(19) United States
(12) Patent Application Publication
(10) Pub. No.: US 2015/0125921 A1
(43) Pub. Date: May 7, 2015

(54) USE OF ANAEROBIC DIGESTION TO DESTROY ANTIBIOTICS IN ORGANIC WASTE

(71) Applicant: Himark bioGas Inc., Vegreville (CA)

(72) Inventors: Bern Kotelko, Vegreville (CA); Tiejun Gao, Edmonton (CA)

(73) Assignee: HIMARK BIOGAS INC., Vegreville, AB (CA)

(21) Appl. No.: 14/400,677

(22) PCT Filed: Mar. 15, 2013
(86) PCT No.: PCT/CA2013/050215

§ 371 Date: Nov. 12, 2014

Related U.S. Application Data
(60) Provisional application No. 61/614,836, filed on Mar. 23, 2012.

(51) Int. Cl. A62D 3/02; B09B 3/00; C12P 5/02; C02F 11/04

(52) U.S. Cl. CPC A62D 3/02; C02F 11/04; B09B 3/00; C12P 5/023; A62D 2101/20
USPC 435/167

(57) ABSTRACT

The invention relates to systems and methods for using the anaerobic digestion (AD) process, especially thermophilic anaerobic digestion (TAD), to destroy biohazard materials including antibiotics.

### REFERENCE TO RELATED APPLICATIONS

This application claims the benefit of the filing date of U.S. Provisional Application No. 61/614,836, filed on Mar. 23, 2012, the entire content of which, including the specification and the drawings, is incorporated herein by reference.

This application is also related to U.S. Provisional Application Nos. 61/216,733, filed on May 21, 2009, 61/216,746, filed on May 21, 2009, and 61/297,063, filed on Jan. 21, 2010, and U.S. Ser. No. 12/782,208, filed on May 18, 2010, the entire content of each of which, including the specifications and the drawings, are hereby incorporated by reference.

### BACKGROUND OF THE INVENTION

Many protein-based bio-hazardous materials constitute a major health problem world-wide. One of the major categories of such materials includes viruses.

For example, influenza virus is a member of the Orthomyxoviruses causing wide-spread infection in the human respiratory tract, but existing vaccines and drug therapy are of limited value. In a typical year, 20% of the human population is afflicted by the virus, resulting in 40,000 deaths. In one of the most devastating human catastrophes in history, at least 20 million people died worldwide during the 1918 Influenza A virus pandemic. The threat of a new influenza pandemic persists because existing vaccines or therapies are of limited value. In elderly the efficacy of vaccination is only about 40%. The existing vaccines have to be redesigned every year, because of genetic variation of the viral antigens, the Haemagglutinin HA and the Neuraminidase N. Four antiviral drugs have been approved in the United States for treatment and/or prophylaxis of influenza. However, their use is limited because of severe side effects and the possible emergence of resistant viruses.

In the U.S., the major cause of diarrhea is virus infections, such as norovirus, rotavirus and other enteric viruses.

HIV (formally known as HTLV-III and lymphadenopathy-associated virus) is a retrovirus that is the cause of the disease known as AIDS (Acquired Immunodeficiency Syndrome), a syndrome where the immune system begins to fail, leading to many life-threatening opportunistic infections. HIV has been implicated as the primary cause of AIDS and can be transmitted via exposure to bodily fluids. In addition to percutaneous injury, contact with mucous membranes or non-intact skin with blood, fluids containing blood, tissue or other potentially infectious body fluids pose an infectious risk.

Many of these infectious viral agents, after coining into contact with certain biological materials, such materials become biohazard. Most (if not all) of these biohazard materials require a proper disposal.

Other protein-based bio-hazardous materials include prion, which may be present in so-called “specified risk materials (SRM).” Management of SRM, such as SRM from cattle (as a potential BSE prion source), is still a global challenge. A cost-effective and environmentally responsible way to destroy prions and utilize decontaminated SRMs is highly desirable for the cattle industry.

BSE has been one of the biggest economic and social challenges to world's beef industry. In Canada alone, BSE caused a loss of over $6 billion since May of 2003. Transmissible spongiform encephalopathies (TSEs) form a group of fatal neurodegenerative disorders represented by Creutzfeldt-Jakob disease (CJD), Gerstmann-Sträussler-Scheinker syndrome (GSS), and Fatal familial insomnia (FFI) in humans; and by scrapie, chronic wasting disease (CWD) and bovine spongiform encephalopathy (BSE) in animals (Collinge, 2001). Evidence accumulated during the major RSE epizootics in the UK (Belay et al., 2004) has confirmed a link between BSE and CJD. One critical step in preventing human infection is to eliminate the pathogen from the food chain and the environment, because transmission routes and mechanisms are not fully understood.

Prions are thought to be the pathogens causing TSEs. Prions, PrP^{sc}, are primarily comprised of a proteinase-K-resistant mis-folded isoform of the cellular prion protein PrP^{c} (Prusiner, 1998). Prions are resistant to inactivation methods usually effective against many microorganisms (Millson et al., 1976; Chatigny and Prusiner, 1979; and Taylor 1991, 2000). A number of studies have reported that chemical disinfection (Brown et al., 1982), autoclaving at 121° C. for 1 hr (Brown et al., 1986, Taylor et al., 1997), exposure to 6 M Urea and 1 M NaOH (Brown et al., 1984, 1986), treatment with 1M NaSCN (Prusiner et al., 1981) and 0.5% hypochlorite (Brown et al., 1986), exposure to sodium hyperchlorite up to 14,000 ppm (Taylor, 1993), digestion with proteinase K (Kocisko et al., 1994; Caughey et al., 1997) and other newly identified proteases (McLeod et al., 2004; Langeveld et al., 2003) could not completely destroy the PrP^{sc}. Inactivation of PrP^{sc} in renderings has been evaluated in the UK and Europe (Taylor and Woodgate, 2003).

Enzymatic degradation of PrP^{sc} has also been studied as a means to achieve decontamination and reuse of contaminated equipment. For example, using the Sup35 Nm-His6 recombinant prion protein to represent the BSE prion, Wang showed that surrogate BSE was selectively digested by subtilisin and keratinase but not by collagenase and elastases (Wang et al., 2005). Six strains of bacteria from 190 protease-secreting isolates were reported o produce proteases which exhibited digestive activities against PrP^{sc} (Müller-Hellwig et al., 2006). Some thermostable proteases produced by the bacteria degraded PrP^{sc} at high temperature and pH 10 (Hui et al., 2004; McLeod et al., 2004; Tsiroulnikov et al., 2004; Yoshioka et al.).

So far, however, incineration is the only effective method to completely destroy prion. But incineration has certain undesirable ecological disadvantages, particularly energy consumption and green house gas emissions. For example, although the CFIA (Canadian Food and Inspection Agency) sanctions only incineration, alkaline hydrolysis and thermal-hydrolysis methods for the safe disposal of SRMs, incineration seems impractical for handling SRMs, especially in large scale, partly because of the industry's lack of capacity and the high associated costs. The limited capacity of existing incinerators and alkaline or thermal hydrolysis facilities, combined with the cost burden of carrying out these processes for destroying SRMs create onerous challenges to the livestock industry. It is estimated that 50,000 to 65.000 tons of SRMs are produced in Canada annually (Facklam, 2007). Incineration of SRMs consumes not only energy but also emits significant amounts of green house gas. In addition, end-products from these procedures are not useful for production of value-added byproducts.

There is growing public concern for the presence of hormones and antimicrobials in water and soil, and the possible pathways by which these substances can enter into human and animal food chains. Of key concern is the routine administration of these substances in livestock farming and concentrated feedlot practices. Antimicrobials are used at both therapeutic and sub-therapeutic levels for disease prevention, control, and treatment. They also serve to improve nutritional efficiency and promote growth rate. Hormones are administered as reproductive regulators and growth promoters. Recent estimates suggest that of the order of 16 million kilograms of hormones and antimicrobials are used annually in routine agricultural operations in North America. It is estimated that up to 80% of administered hormones and antimicrobials are excreted in unaltered form, or as partially metabolized derivatives that retain their pharmaceutical activity.

A 2011 survey conducted by the United States Department of Agriculture reported that there were 105 million head of cattle in North America, producing upwards of one billion kilograms of manure waste annually. A significant portion of these cattle are raised in confined animal feeding operations (CAFOs), where manure is land applied or composted. A growing body of evidence suggests that hormones and antimicrobials can persist for prolonged periods in manure and, given the land application of cattle waste as a source of fertilizer, transfer to surface and groundwater is likely. Antimicrobial residues in the environment are of concern due to the development of drug-resistant strains amongst native microbial communities in soil.

Hormones can act as endocrine disrupting chemicals (EDCs) which pose significant harmful impacts to wildlife, particularly birds and fish. Whilst the fate of antimicrobials under conditions of composting or in lagoons has been documented, there is little information on the effect of anaerobic digestion (AD) on degradation of residual hormones and antimicrobials in manure and Feedlot wastes.

Tylosin (TYL), chlortetracycline (CTC) and sulfamethazine (SMZ) represent the most widely used, broad spectrum antimicrobials administered at sub-therapeutic doses for cattle. They also represent the structural diversity of different antimicrobial classes. Megestrol (MEG) is a synthetic gestagen (an exogenous hormone). The structures of the antimicrobials and exogenous hormones are shown below.

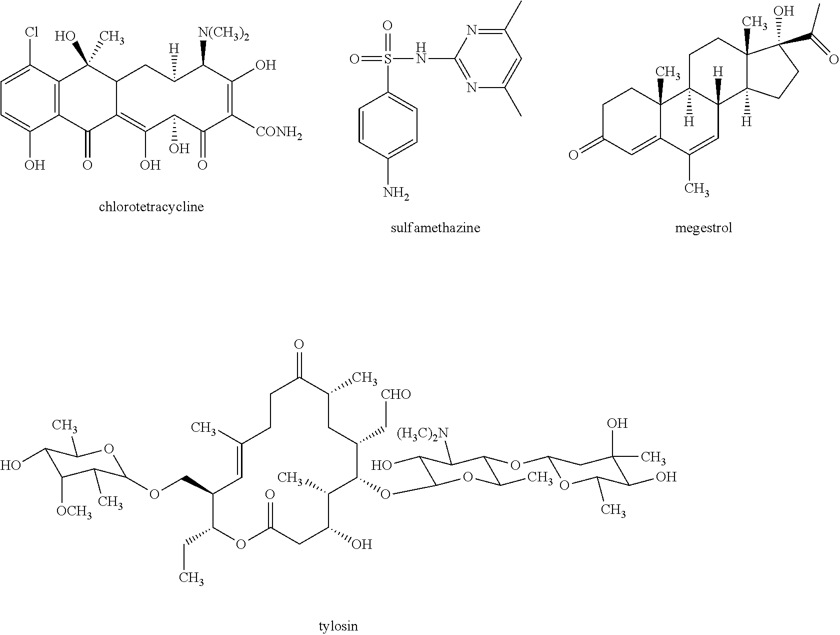

In summary, certain antibiotics and exogenous hormones are used in human and animal treatment, e.g., in vet medicine. Such antibiotics may be present in certain biological materials as biowaste material, which can unexpectedly leak into animal feed or other food sources, leading undesired or unexpected consequences. How to properly dispose of biological materials or biowaste that may contain such antibiotics could be an environmental challenge.

### SUMMARY OF THE INVENTION

One aspect of the invention provides a method for reducing the titer of a biohazard that may be present in a carrier material, comprising providing the carrier material to an anaerobic digestion (AD) reactor and maintaining the rate of biogas production substantially steady during the AD process.

In certain embodiments, the biohazard comprises antibiotics, exogenous hormones, antibodies, body fluids (e.g., blood), viral pathogens, bacterial pathogens, and/or weed seeds. In other embodiments, the biohazard comprises prion. For example, the prion may be scrapie prion, CWD prion, or BSE prion. The prion may be resistant to proteinase K (PK) digestion.

In certain embodiments, the invention provides a method for accelerating and/or enhancing degradation of an antibiotic or an exogenous hormone that may be present in a carrier material (such as an organic waste), comprising providing the carrier material to an anaerobic digestion (AD) reactor, and optionally maintaining the rate of biogas production substantially steady during the AD process.

As used herein, “to accelerate degradation” includes increasing the rate certain compounds, such as antibiotics or hormones, are degraded under certain conditions (such as TAD), as compared to the rate of degradation in the absence of the condition.

As used herein, “to enhancing degradation” includes increasing the rate and/or extend of degradation. For example, an antibiotic may not normally degrade completely (about 100%) or mostly (e.g., >50%), but enhanced degradation may degrade up to 80%, 90%, 95%, 99% or nearly 100% of the antibiotic, and may reach that enhanced level or extent of degradation faster.

The carrier material may be an organic waste, such as one that support methane production through anaerobic digestion. The organic waste may include animal meat or offal or other biological waste from slaughter house, animal manure, other animal body fluid or excretes, food waste, municipal waste, sewage, etc.

In certain embodiments, the carrier material is a biowaste material (e.g., animal meat, animal manure, or other animal body fluid or excretes), or wet distilled grains with solubles (WDGS) from biorefinery.

In certain embodiments, antibiotics include those used in veterinary medicine, or human medicine. For example, chlortetracycline, Tylosin, sulfamethazine, and similar or related antibiotics.

Chlortetracycline related antibiotics include tetracycline and the related group of broad-spectrum antibiotics with four hydrocarbon rings, more specifically defined as a subclass of polyketides having an octahydrotetracene-2-carboxamide skeleton. They are collectively known as derivatives of polycyclic naphthacene carboxamide. Specific examples of such tetracycline related antibiotics include: naturally occurring antibiotics such as Tetracycline, Chlortetracycline, Oxytetracycline, Demeclocycline and semi-synthetic antibiotics such as Doxycycline, Lymecycline, Meclocycline, Methacycline, Minocycline, and Rolitetracycline. Additional such antibiotics include PTK0796, and Tigecycline (and its related glycylcycline antibiotics).

Tylosin and its related macrolide-class antibiotic, such as those used in veterinary medicine, include a group of antibiotics whose activity stems from the presence of a macrolide ring, a large macrocyclic lactone ring to which one or more deoxy sugars, usually cladinose and desosamine, may be attached. The lactone rings are usually 14-, 15-, or 16-membered. Macrolides belong to the polyketide class of natural products. Antibiotic macrolides include US FDA-approved drugs, such as Azithromycin, Clarithromycin, Dirithromycin, Erythromycin, Roxithromycin, and Telithromycin; non-US FDA-approved drugs such as Carbomycin A, Josamycin, Kitasamycin, Midecamycin/midecamycin acetate, Oleandomycin, Solithromycin, Spiramycin (approved in Europe and other countries), Troleandomycin (used in Italy and Turkey), Tylosin/tylocine (used in animals).

Other Tylosin related antibiotics include Ketolides, a new class of antibiotics that are structurally related to the macrolides. Ketolides include: Telithromycin, Cethromycin, Solithromycin (the first fluoroketolide), Spiramycin (used for treating toxoplasmosis), Ansamycin, Oleandomycin, and Carbomycin.

Sulfadimidine (or sulfamethazine, sulfadimerazine, sulfadimezine, and sulphadimethylpyrimidine) is a sulfonamide antibacterial agent that includes synthetic antimicrobial agents containing the sulfonamide group. Antibiotic sulfonamides include short-acting antibiotics such as Sulfamethoxazole and Sulfisomidine (also known as sulfaisodimidine); intermediate-acting antibiotics such as Sulfacetamide and Sulfadoxine; and Ophthalmologicals such as Dichlorphenamide (DCP) and Dorzolamide.

Other related sulfonamides are diuretics such as Acetazolamide, Bumetanide, Chlorthalidone, Clopamide, Furosemide, Hydrochlorothiazide (HCT, HCTZ, HZT), indapamide, Mefruside, Metolazone, Xipamide, Anticonvulsants, Acetazolamide, Ethoxzolamide, Sultiame, Zonisamide, Dermatologicals, and Mafenide.

Other sulfonamides include Celecoxib (COX-2 inhibitor), Darunavir (Protease Inhibitor), Probenecid (PBN), Sulfasalazine (SSZ), and Sumatriptan (SMT).

In certain embodiments, the antibiotics do not substantially degrade by high temperature alone (e.g., incubation at about 50-60° C., or 55° C.).

In certain embodiments, the antibiotic is a sulfonamide antibacterial agent that includes a sulfonamide group (e.g., sulfamethazine).

In certain embodiments, the antibiotic is a veterinary medicine.

In certain embodiments, the antibiotic is present in a biowaste material (e.g., animal meat, animal manure, or other animal body fluid or excretes).

In certain embodiments, the exogenous hormone is a steroidal progestin, such as megestrol or megestrol acetate.

In certain embodiments, the exogenous hormone is not a polypeptide, or does not comprise a polypeptide or amino acid residue.

In certain embodiments, the AD reactor may be operated in batch mode. The batch mode may last less than about 0.5 hr, 1 hr, 2 hr, 5 hr, 10 hr, 24 hr, 2 days, 3, 4, 5, 6, 7, 10, 20, 30, 40, 50, or 60 days. For viral and bacterial agents, the batch mode generally lasts from less than about a few hours to several days (e.g., 1-7 days), depending on temperature used. For especially stable agents, such as prion, the batch mode generally lasts less than about 30, 40, 50, or 60 days.

In other embodiments, the rate of biogas production peaks at about 0.5-5 hrs, 1-7 days, or 5-10 days after the beginning of the batch mode operation.

In other embodiments, it may be operated in semi-continuous mode, or continuous mode.

In certain embodiments, the AD reactor contains an active inoculum of microorganisms at the beginning of the batch mode operation.

In certain embodiments, the AD process is carried out by a consortium of anaerobic microorganisms, such as psychrophilic microorganisms (e.g., those with optimal growth conditions around 20° C. or so), mesophilic microorganisms (e.g., those with optimal growth conditions around 37° C. or so), or thermophilic microorganisms (e.g., those with optimal growth conditions above 45-48° C. or so, such as 55° C., 60° C., 65° C.).

In certain embodiments, the thermophilic microorganisms are acclimatized with substrates containing proteins with abundant β-sheets. This may be helpful for removing bio-hazard materials.

In certain embodiments, the thermophilic microorganisms are acclimatized by culturing with substrates containing amyloid substance at elevated temperature and extreme alkaline pH. The period can last, for example, for 3 months.

In certain embodiments, the method further comprises adding one or more supplemental nutrients selected from Ca, Fe, Ni, or Co.

In certain embodiments, the AD is carried out at about 20° C., 25° C., 30° C., 37° C., 40° C., 45° C., 50° C., 55° C., 60° C. or above.

In certain embodiments, >50%, 60%, 70%, 80%, 90% or more (in terms of amount) of the antibiotic is degraded after about 2 clays to 2 weeks of anaerobic digestion, about 2 days to 1 week of anaerobic digestion, or about 3-5 days of anaerobic digestion.

In certain embodiments, more than 50%, 60%, 70%, 80%, 85%, 90%, 95%, 99%, or nearly 100% of the antibiotic in the carrier material is degraded.

In certain embodiments, the rate of biogas production is reduced by no more than 40%, 30%, 25%, 20%, 15%, 10%, 5% or less.

In certain embodiments, the rate of methane production is reduced by no more than 25%, 20%, 15%, 10%, 5% or less.

It is contemplated that all embodiments described herein, including embodiments described separately under different aspects of the invention, can be combined with features in other embodiments whenever applicable.

### BRIEF DESCRIPTION OF THE DRAWINGS

FIG. 1 shows results when scrapie-containing and normal sheep brain homogenates were spiked in TAD (thermophilic anaerobic digestion) digester, and incubated for a set period of time. The numbers 1 to 4 indicated different sampling times post digestively. The protein from the TAD-tissue mixtures at different time points was isolated, purified, and resolved by 12.5% SDS-PAGE gel, and subjected to Western blotting detection with ECL substrate. Large amounts of prion proteins were recovered from TAD sludge before digestion (time 0). In contrast, none was found in TAD control without the tissues. Cellular prion had disappeared at sampling time 1 (TAD-normal sheep brain mix), but scrapie was completely eliminated at sampling time 2 (TAD-scrapie mix). The 27 kDa protein marker indicates mobility of sheep cellular prion and scrapie prion.

FIG. 2 demonstrates protein-load dependent methanation in the pilot study of scrapie inactivation during the course of TAD. TAD was set up with the same amount of the digestate containing different amounts of scrapie-infected sheep brain tissue and normal sheep brain tissue (in low dose and high dose, respectively). TAD alone was used as control. The highest volume of methane production was achieved in high-dose protein load groups (scrapie and normal sheep brain), and then in low-dose protein load groups (scrapie and normal sheep brain), in comparison with the control one. It indicates clearly that an increase of protein load at a given level in TAD enhances biogas production and CH₄/CO₂ ratio, thus increases fuel value of biogas.

FIG. 3 shows assessment strategy for post-digest Scrapie prion samples in anaerobic digestion.

FIG. 4 is a summary of time- and dose-dependent viral inactivation based on assessment of viral infection on cultured cells (cytopathic effect, CPE %).

FIG. 5 demonstrates that Scrapie prion (S. prion) showed different degrees of reduction in the presence of absence of additional cellulosic substrates in TAD digestion processing at day 11, 18 and 26. The image was quantified using Alpha Innotech Image analyzer.

FIG. 6 shows that degradation of selected antibiotic/hormone in: (a) group 1, Active TAD (manure and AD inoculums), 55° C.; (b) group 2, fresh manure and water control group, 55° C.; (c) group 3, sterile water control, 55° C.; (d) group 4, sterile water control, 22° C.

FIGS. 7A-7D show degradation profiles of 20 ppm hormone/antimicrobials under various conditions (see Table 4): (7A) tylosin; (7B) chlorotetracycline; (7C) sulfamethazine; (7D) megestrol.

FIGS. 8A-8C show accumulated biogas (8A), methane (8B), and CO₂ (8C) production from digesters in different groups during 28 days.

### DETAILED DESCRIPTION OF THE INVENTION

The invention is partly based on the discovery that destruction of certain biohazards (such as antibiotics) occurs in an active stage of anaerobic digestion (AD). Such biohazards may be present in a carrier material, and may include weed seeds, certain protein-rich pathogens or undesirable pertinacious materials (e.g., hormones, antibodies, viral pathogens, body fluids (e.g., blood), bacterial pathogens, etc.), or prions within a specified risk material (SRM). While not wishing to be bound by any particular theory, it is contemplated that at high biogas production rate, microbial activity is high or microbial growth rate is high, thus increasing the chance and/or rate of breaking down such biohazards.

The invention is also partly based on the discovery that certain small molecules within the anaerobic digestion (AD) system, especially the TAD system, may inactivate at least certain viral infectious agents. Thus such molecules, either purified or unpurified from the liquid anaerobic digestate, may be used to inactivate viral agents.

The invention is further based on the discovery that adding a carbohydrate-based substrate (such as cellulose or cellulose type material) periodically to the digester may accelerate or enhance the reduction of pathogen titer. The carbohydrate-based substrate may be added at a w/v percentage of about 0.5%, 1%, 1.5%, 2%, 2.5%, 3%, 4%, 5%, 8%, 10%, 15%, or between any of the two referenced values (as measured by the weight (in gram) of the carbohydrate-based substrate over volume (in mL) of the digestate). One or more additions of the carbohydrate-based substrate may be made during the period of digestion. The intervals of adding the carbohydrate-based substrate may be substantially identical (e.g., about 7-8 days between additions) or different. The timing of addition preferably substantially coincides with the biogas production rate, e.g., just prior to or around the time peak biogas production is expected to dip.

Therefore, in one aspect, the invention provides a method for reducing the titer, amount, or effective concentration of a biohazard that may be present in a carrier material, comprising providing the carrier material to an anaerobic digestion (AD) reactor and maintaining the rate of biogas production substantially steady during the AD process after biogas production has reached a peak rate. The AD reactor may be operated in batch mode, semi-continuous mode, or continuous mode.

In a specific embodiment, the invention provides a method for accelerating and/or enhancing degradation of an antibiotic or steroidal hormone (e.g., steroidal progestin) that may be present in a carrier material (such as an organic waste), comprising providing the carrier material to an anaerobic digestion (AD) reactor, preferably under TAD condition. Optionally, the rate of biogas and/or methane production is substantially steady during the AD process, or the rate of methane production is reduced by no more than 20%, 15%, 10%, 5% or less.

In certain embodiments, the concentration of the antibiotics in the AD reactor is no more than 100 ppm, 90 ppm, 80 ppm, 70 ppm, 60 ppm, 50 ppm, 40 ppm, 30 ppm, 20 ppm, 10 ppm, 5 ppm, 4 ppm, 3 ppm, 2 ppm, 1 ppm or less.

Rate of gas production may be measured in any of the industry standard methods, so long, as a consistent method is used for monitoring gas production rate. Suitable methods include measuring gas pressure, gas flow rate, etc. Methane to carbon dioxide ratio may also be used for this purpose. Other techniques, such as gas chromatography (GC) that measures gas quality, in combination with gas flow and gas pressure measures, may be used to determine the production of methane and other biogas components.

Almost any biohazard materials agents can be the target of the subject method, including antibiotics, bacterial pathogens (e.g., E. coli, Salmonella, listeria), viral pathogens (e.g., HIV/AIDS, picornavirus such as foot-and-mouth disease virus (FMDV), equine infectious anemia virus, porcine reproductive and respiratory syndrome virus (PRRSV), also known as Blue-Ear Pig Disease, porcine circovirus type 2, bovine herpesvirus 1, Bovine Viral Diarrhea (BVD), Border Disease virus (in sheep), and swine fever virus), parasitic pathogens, prions, undesirable hormones, blood and other body fluids.

One particular type of biohazard, prion (scrapie prion, CWD prion, or BSE prion, etc.), is of particular interest. Such prion may be resistant to proteinase K (PK) digestion, and may be present in a protein-rich carrier material, such as a specified risk material (SRM).

As used herein, “specified risk material” is a general term referring to tissues originating from any animals of any age that potentially carry and/or transmit TSE prions (such as BSE, scrapie, CWD, OD, etc.). These can include skull, trigeminal ganglia (nerves attached to brain and close to the skull exterior), brain, eye, spinal cord, CNS tissue, distal ileum (a part of the small intestine), dorsal root ganglia (nerves attached to the spinal cord and close to the vertebral column), tonsil, intestine, vertebral column, and other organs.

As used herein, “batch mode” refers to the situation where no liquid or solid material is removed from the reactor during the AD process. Preferably, the feedstock and other materials necessary for the AD process are provided to the reactor at the beginning of the batch mode operation. In certain embodiments, however, additional materials may be added to the reactor.

In contrast, in continuous mode or semi-continuous mode, solids and liquids are being continuously or periodically (respectively) removed from the AD reactor.

For example, the AD reactor may contain an active inoculum of microorganisms, e.g., at the beginning of the batch mode operation. The active inoculum of microorganisms may be obtained from the previous batch of operation, with optional dilution to adjust the proper volume of the inoculum and the feedstock in the AD reactor. One associated advantage is that the microorganisms within the inoculum are already primed to produce biogas at optimal rate at the beginning of the operation, such that peak biogas production rate can be achieved in a relatively short period of time, e.g., between about 5-10 days.

Due to the natural fluctuation attic biogas production rate, “substantially steady” means that the biogas production rate generally does not deviate from the average value by more than 50%, preferably no more than 40%, 30%, 20%, 10%, or less. Substantially steady gas production rate can be maintained by periodically adding to the anaerobic digestion reaction suitable amounts of additional substrates, preferably those do not contain significant amount of pathogens to be destroyed (in the batch mode operation), at a time around the time point when peak or plateau gas production rate is about to decline.

In certain embodiments, a carbon-rich material may also be provided, semi-continuously to the AD reactor once every about 5-10 days after reaching peak biogas production, to maintain substantially steady biogas production. There are many suitable carbon-rich materials that can be used in the instant invention. In certain embodiments, the carbon-rich material may comprise fresh plant residues or other easily digestible cellulose.

The AD process is preferably carried out under thermophilic conditions, and such thermophilic anaerobic digestion (or “TAD”) is shown to efficiently eliminate various biohazard materials such as SRMs (Specified Risk Materials), including materials containing various prion species. TAD provides several advantages for SRM destruction, including its thermo-effect, a hydraulic batch of homogeneous system with high pH, synergistic effects of enzymatic catalysis, volatile fatty acids, and/or biodegradation of anaerobic bacterial colonies. The TAD process also has the added advantage of allowing SRMs to be safely used as a biomass/feedstock source for the production of biogas and other byproducts.

Thus in certain embodiments, the temperature of the AD reactor is controlled at about 20° C., 25° C., 30° C., 37° C., 40° C., 45° C., 50° C., 55° C., 60° C., or above to facilitate a thermophilic anaerobic digestion (TAD) process. In certain preferred embodiments, the AD process is carried out by a consortium of thermophilic microorganisms, such as thermophilic bacteria or archaea.

Preferably, the starting pH of the TAD process is about 8.0, or about pH 7.5-8.5. pH regulating agents or buffers may be added to the reactor periodically, if necessary, to control the pH at a desired level throughout the AD process.

In certain situations, conventional TAD may or may not completely destroy prion or other biohazards/pathogens, possibly because of the lack of essential anaerobic bacterial colonies and enzymes required for the specific catalysis. Thus in certain situations, the anaerobic microorganisms may be acclimatized so that they are more adapted to destroying the intended target. For instance, in the case of prion, acclimatization can be done using substrates containing proteins with abundant β-sheets. For example, selected anaerobic digestates may be cultured with special substrates containing amyloid substance at elevated temperature and extreme alkaline pH for about 3 months. Cultures using such acclimatized microorganisms may be further optimized by monitoring and adjusting biogas production profile, composition, and total ammonia nitrogen (TAN) to ensure that no inhibition of anaerobic digestion occurs. In certain embodiments, supplemental nutrients (such as Ca, Fe, Ni, or Co) may be added to increase efficient removal of propionate as volatile fatty acid (VFA).

Optionally, genetic evolution of anaerobic microorganism colonies during acclimatization can be analyzed with real-time PCR-based genotyping using specially designed primers and probes. Furthermore, decontamination capability of these acclimatized anaerobic microorganism batches can be tested and compared with conventional TAD in regards to the elimination rate of the prion.

Destruction of any types of viral pathogens may be effectuated by using the subject methods. Exemplary (non-limiting) viral pathogens (or bio-hazardous materials containing such viral pathogens) that may be destroyed using the subject methods include: influenza virus (orthomyxovirus), coronavirus, smallpox virus, cowpox virus, monkeypox virus, West Nile virus, vaccinia virus, respiratory syncytial virus, rhinovirus, arterivirus, filovirus, picorna virus, reovirus, retrovirus, pap ova virus, herpes virus, poxvirus, headman virus, atrocious, Coxsackie's virus, paramyxoviridae, orthomyxoviridae, echovirus, enterovirus, cardiovirus, togavirus, rhabdovirus, bunyavirus, arenavirus, bornavirus, adenovirus, parvovirus, flavivirus, norovirus, rotavirus, and other enteric viruses. Other viral pathogens include those detrimental to animal health, especially those found in and responsible for various viral diseases of the livestock animals. Such viruses may be present in disease tissues of livestock animals.

Destruction of any types of bacterial pathogens may be effectuated by using the subject methods. Exemplary (non-limiting) bacterial pathogens (or bio-hazardous materials containing such bacterial pathogens) that may be destroyed using the subject methods include: bacteria that cause intestine infection, such as E. coli (particularly enterotoxigenic E. coli and E. coli strain O157:H7), which bacteria cause stresses for municipal wastewater treatment; bacteria that cause food-related outbreaks of listerosis, such as Listeria M.; bacteria that cause bacterial enterocolitis, such as Campylobacter jejuni, Salmonella EPEC, and Clostridium difficile.

Destruction of any types of parasitic pathogens may be effectuated by using the subject methods. Exemplary (non-limiting) parasitic pathogens (or bio-hazardous materials containing such parasitic pathogens) that may be destroyed using the subject methods include: Giardia lamblia and Crytosporidium.

Fungal or yeast pathogens can also be eliminated by the subject method.

Any of the pathogen containing materials may be used in the methods of the instant application. For example, in certain hospitals (including vet hospitals) or healthcare facilities, patient (human or non-human animal) stools and/or body fluids (e.g., blood) may be rich sources of viral, bacterial, and/or parasitic pathogens that should be decontaminated before releasing to the public water or waste disposal. Such bio-waste materials may be used as carrier materials for the methods of the invention.

Destruction of numerous types of prions may be effectuated by using the subject methods. As used herein, “prion” includes all infectious agents that cause various forms of transmissible spongiform encephalopathies (TSEs) in various mammals, including the scrapie prion of sheep and goats, the chronic wasting disease (CWD) prion of white-tailed deer, elk and mule deer, the BSE prion of cattle, the transmissible mink encephalopathy (TME) prion of mink, the feline spongiform encephalopathy (FSE) prion of cats, the exotic ungulate encephalopathy (EUE) prion of nyala, oryx and greater kudu, the spongiform encephalopathy prion of the ostrich, the Creutzfeldt-Jakob disease (CJD) and its varieties prion of human (such as iatrogenic Creutzfeldt-Jakob disease (iCJD), variant Creutzfeldt-Jakob disease (vCJD), familial Creutzfeldt-Jakob disease (fCJD), and sporadic Creutzfeldt-Jakob disease (sCJD), the Gerstmann-Sträussler-Scheinker (GSS) syndrome prion of human, the fatal familial insomnia (FFI) prion of human, and the kuru prion of human.

Certain fungal prion-like proteins may also be destroyed, if necessary, using the subject methods. These include: yeast prion (such as those found in Saccharomyces cerevisiae) and Podospora anserina prion.

The amount of prions or other biohazards/proteinaceous pathogens used in the subject method can also be adjusted. In certain embodiments, an equivalent of about 1-10 g, or about 2.5-5 g of prion-containing tissue homogenate is present in every about 60 to 75 ml of TAD-tissue mixture. For TAD-tissue mixture having protein load towards the high end of the range, about 1 g of carbon-rich material (e.g., cellulose) may be added according to the scheme described herein to every about 60-75 mL of TAD-tissue mixture.

Destruction of any types of antibiotics and/or steroidal progestin may be effectuated by using the subject methods. In certain embodiments, antibiotics include those used in veterinary medicine, or human medicine. For example, chlortetracycline, Tylosin, sulfamethazine, and similar or related antibiotics, such as those described hereinabove.

In certain embodiments, the AD reactor contains at least about 5, 6, 7, 8, or 9% final total solid components.

In certain embodiments, the prion is resistant to proteinase K (PK) digestion.

In certain embodiments, the SRM comprises CNS tissue, such as tissues from brain, spinal cord, or fractions, homogenates, or parts thereof.

In certain embodiments, the batch mode operation lasts less than about 20, 30, 40, 50, 60, 70, 80, 90, 100, 110, or 120 days. At the end of the batch mode operation, the titer of the biohazard/prion is reduced by at least about 2, 3, or 4 logs. For example, in certain embodiments, 2 logs or more reduction of the titer of the biohazard/prion is achieved after about 60, 30, or even 18 days of anaerobic digestion. In certain other embodiments, 3 logs or more reduction of the titer of the bio-hazard prion is achieved after about 20, 25, 30, 35, 40, 45, 50, 55, 60 or more days of thermophilic anaerobic digestion. In certain embodiments, 4 logs or more reduction of the titer of the bio-hazard prion is achieved after about 30, 40, 50, 60, 70, 80, 90 or more days of thermophilic anaerobic digestion.

The invention is also partly based on the discovery that enhanced biogas (e.g., methane or CH₄) production through anaerobic digestion can be achieved by using a protein-rich feedstock. Furthermore, biogas production may be further enhanced by semi-continuously providing a carbon-rich material, optionally together with additional protein-rich material, to the AD reactor in order to maintain the rate of biogas production substantially steady during the AD process, preferably also with high quality (i.e., CH₄ higher than 50, 55, 60, 65, or 70%). While not wishing to be bound by any particular theory, the observed enhanced biogas production suggests that the AD process allows various microorganisms present in the AD bioreactor to breakdown the protein-rich feedstock to supply nitrogen and/or carbon for microbial growth, and ultimately methane production (i.e., methanogenesis is highly efficient).

Thus in one aspect, the invention provides a method for producing biogas, preferably with higher fuel value and high quality, comprising providing to an anaerobic digestion (AD) reactor a protein-rich feedstock, wherein the rate of biogas production is maintained substantially steady during the AD process after a peak rate of biogas production is reached.

In certain embodiments, the AD reactor may be operated in batch mode. In other embodiments, the AD reactor may be operated in continuous or semi-continuous mode, with continuous or periodic addition and removal of solids/liquids from the reactor during the AD process.

Regardless of the operational mode, a carbon-rich material may be provided to the reactor during the AD process to sustain the peak rate of biogas production. For example, in the batch mode, the carbon-rich material may be semi-continuously or periodically provided to the AD reactor once every about 5-10 days after reaching peak biogas production rate, in order to maintain substantially steady biogas production. Such carbon-rich material may include fresh plant residues, or any other easily digestible cellulose. In continuous or semi-continuous mode operation, the carbon-rich material and optionally the protein-rich feedstock may be added either together or sequentially/alternatively to sustain steady state biogas production.

In certain embodiments, the hatch mode operation may last less than about 30, 40, 50, 60, 70, 80, 90, 100, 110, or 120 days.

In certain embodiments, the biogas fuel value, as defined by the ratio of methane over CO₂, is roughly directly proportional to (or otherwise positively correlated with) the protein content in the feedstock. Under optimal conditions, protein degradation occurs rapidly during the first 5-10 days of the AD process. During this period, peak protein degradation coincides with peak biogas production rate.

Almost any protein-rich feedstock can be used for the instant invention. In certain embodiments, the protein-rich feedstock is a specified risk material (SRM). For example, the SRM may comprise one or more prions or pathogens. Such SRM may comprise CNS tissues (e.g., brain, spinal cord, or fractions/homogenates/parts thereof). Prions may include scrapie, CWD, and/or BSE prions, etc. (supra). In certain embodiments, the prions are resistant to proteinase K (PK) digestion. Batch mode is preferred if SRM containing prion is used as the protein-rich feedstock.

In other embodiments, the protein-rich feedstock may comprise hormones, antibodies, viral pathogens, or bacterial pathogens, or any other proteinaceous substance.

Another aspect of the invention provides a protein extraction method to achieve the maximal recovery of prion proteins from anaerobic digestate. This method can be used, either alone or in conjunction with traditional biochemistry techniques (such as Western blotting (WB) and any commercialized BSE-Scrapie Test kit, etc.), to examine and document the elimination rate of prions during and after the TAD process. Preferably, a series of positive controls may be included in the assay.

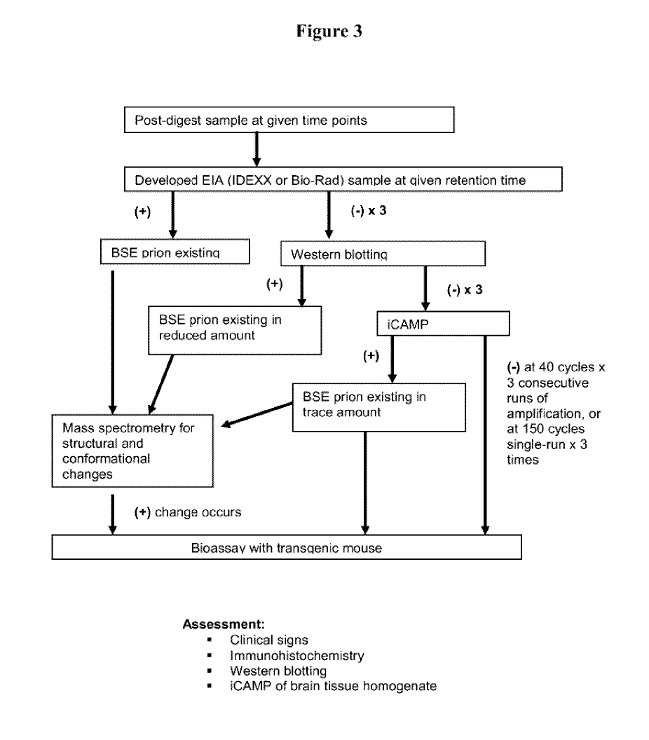

Another aspect of the invention provides a method to determine the presence and/or relative amount of residual prions in the post-digestion sample. The method may comprise one or more technologies useful for prion detection, or combinations thereof. In a preferred embodiment, as shown in FIG. 3, post-digestion sample obtained at any given time points during the AD process may be subjected to successive rounds of analysis including EIA, Western Blotting (WB), iCAMP, and bioassay with transgenic mouse, progressing to the next level of (more sensitive but expensive/difficult slower) analysis only when the previous level of (less sensitive but cheaper/easier/faster) analysis has failed to confirmed the absence of prion in the sample.

For example, if EIA is sufficient to detect the presence of prion, there will be no need to run more complicated assays to confirm the existence of prion. Only when EIA Fails to detect prion would WB become necessary for the next level of analysis.

Similarly, in certain embodiments, when WB fails to detect prion after multiple tests, a highly sensitive detection method termed in vitro cyclic amplification of mis-folding protein (iCAMP) may be used to verify the absence of prion (thus the completion of prion destruction) in the TAD discharge. In certain embodiments, a repeatedly negative iCAMP sample can in turn be examined with, for example, a mouse-based bioassay to determine a biologically safe end-point of prion decontamination and to ensure zero-discharge of any prions into the environment.

These prion detection methods are well known in the art. See Groschup and Buschmann, Rodent Models for Prion Diseases, Vet. Res. 39: 32, 2008 (incorporated herein by reference). For example, there are several transgenic mouse models (e.g., Tg 20) that can be used to verify the infectivity and transmission of prion/scrapie before and after AD inactivation. Most of such transgenic mice in prion research are knock-out mice, with their endogenous prion genes knocked out. They generally have increased susceptibility to prion pathogens, including prion pathogens from a different species. Symptoms of prion manifestation—pathological changes in the brain tissue of the affected animals—may be detected or verified using immuno-histochemistry methods, which is one of the most confirmative assays for diagnosis of prion diseases.

For example, US 2002-0004937 A1 describes such a transgenic mouse model for prion detection, comprising introducing a prion gene of an animal (e.g., that of human, cattle, sheep, mouse, rat, hamster, mink, antelope, chimpanzee, gorilla, rhesus monkey, marmoset and squirrel monkey, etc.) into a mouse (preferably a mouse with its endogenous prion genes knocked out) to produce a prion gene modified mouse, and determining that the prion gene is aberrant when the prion gene modified mouse exhibits heart anomalies. Using this mouse, prion titer before and after AD may be measured by, for example, inoculating the transgenic mouse with a sample (before/after AD), and observing the presence of myocardial diseases in the prion gene modified mouse. Samples spiked with known titers of control prion of the same type may be used in the same experiments to quantitatively measure the prion titers before/after the TAD process of the invention.

More specifically, for use in the instant invention, samples obtained at, for example, day 30 or later (in which no prion proteins may be detectable by Western blot, or “WB”), and filtered for sterilization. Then about 50 to 80 μl (usually less than about 100 μl) of the sterilized sample is injected into the brain of a selected transgenic mouse under anesthesia, with undigested prion/scrapie as control in same strain of mice. Observation days are usually 100 to 150 days after inoculation. Earlier samples taken at earlier time points, such as day 18, 11 or even 6 (when WB may show detectable levels of prion/scrapie) may be used in parallel experiments to determine the time period where AD has substantially eliminated active prion in the sample. This type of bio-assay allows one to determine whether prion/scrapie has lost its infectivity, even though the prion protein itself may still be detectable by WB.

Most suitable transgenic mice are available in the art, including from commercial entities (e.g., Jackson Laboratory).

In certain embodiments, the mechanism of prion inactivation and its conformational alteration in post-digest samples can be investigated using mass spectrometry and other proteomic tools (see FIG. 3), This down-stream research can further expand the general knowledge of prion structure and its related pathogenesis, and provide collaborative opportunities for basic researchers to explore fundamental knowledge of prions and develop drugs for treatment of prion-associated diseases in humans (such as CJD).

Multiple advantages can be realized according to the instant invention. For example, prion (Scrapie or BSE, etc.) and its infectivity can be destroyed completely by the TAD within 30 days, 60 days, or 100 days. Meanwhile, protein-rich SRMs with disinfected prions, instead of being waste materials that require costly treatment for proper disposal, can be utilized by the TAD process to enhance fuel value of biogas in comparison to conventional anaerobic digestion. As a result, multiple social and economical benefits can be simultaneously achieved, including allowing the cattle industry to treat SRMs cost-effectively, meeting certain government mandates, protecting the environment from a possible contamination with prion pathogens, reducing the environmental footprint caused by the disposal of SRM treated by other methods, and at the meantime generating valuable biogas. Thus, thermophilic anaerobic digestion process may well eliminate prions in SRMs effectively via combined enzymatic catalysis and biological degradation by anaerobic bacterial colonies in the system, and turn the protein-rich SRMs into bioenergy and biofertilizers.

### EXAMPLES

The invention having been generally described, the following section provides exemplary experimental designs that illustrate the general principle of the invention. The examples are for illustration purpose only, but not limiting in any respect.

In addition, although some examples below are based on prion proteins, other less stable protein-based bio-hazardous materials, including hormones, antibodies, viral pathogens, bacterial pathogens, and/or weed seeds, etc., are expected to behave similarly, if not identical, in similar experiments.

### Example 1

### Thermophilic Anaerobic Digestion (TAD) Process Eliminates Scrapie Prion and Enhances Biogas Production

Scrapie prion, one of the very resistant prions to proteinase K (PK) digestion, was used as a model in this experiment to demonstrate the effectiveness of the TAD process for prion destruction.

High-(4 g) and low-dose (2 g) of scrapie brain homogenate (20%) were spiked into the lab scale TAD digesters, with temperature set at 55° C. Digestion was allowed to continue in hatch mode for up to 90 days. About 5 mL of the digestate was taken from experimental and control groups at day 0, 10, 30, 60, and 90 for assessing scrapie degradation. Scrapie (PrP^{sc}), obtained from the CFIA National Reference Lab, and cellular prion (PrP^{c}) were recovered from the digestate using a buffer containing 0.5% SDS (recovery rate ˜75 to 82%). Both cellular and scrapie prion were resolved in 12.5% SDS-PAGE gel and detected by immunoblotting using a monoclonal antibody (F89, Sigma). Biogas production was monitored regularly to assess activity of anaerobic bacteria and to evaluate effect of protein-rich substrate on biogas production using micro-gas chromatography (GC).

The results demonstrated that scrapie was degraded in a time-dependent manner. While the cellular prion had disappeared by about day 10, no scrapie band was observed at Day 30 in TAD digesters. It was estimated that at least about 2.0 logs or more reduction of scrapie was achieved in 30 days based on computer-assisted semi-quantitation of immunoblotting images. Meanwhile, biogas production and its fuel value (ratio of methane over CO₂) were enhanced significantly in protein-rich TAD. About 2.6-fold more methane was gained in high-dose protein (384.42±6.54 NmL), and about 1.9-fold in low-dose protein TAD (284.39±2.02 NmL) than that in TAD control without protein (145.93±10.33 NmL) during 90 days' of AD digestion.

The data demonstrates that batch TAD can be effectively used as a biological and environment friendly method to decontaminate prion in SRM, and transform SRM from a biohazard into a safe feedstock for producing biogas and other value-added byproducts. This process not only reduces the environmental footprint of prions, but also generates economic benefit to both the cattle industry and local community.

### Example 2

### Efficacy and Kinetics of BSE Elimination in Batch-TAD Under Optimal Conditions

Bovine brain tissue and other types of SRM tissues (such as spinal cord, lymph nodes or salivary glands) with confirmed BSE are obtained from the CFIA National BSE Reference Lab, and homogenized in phosphate buffered saline (PBS) on ice. A 20% brain homogenate alone or homogenate mixed with other tissues is spiked in diluted digestate (with final total solid of about 7%), which is obtained fresh from the IMUS™ demonstration plant in Vegreville, based on results of the studies described above. The whole procedure is carried out in a biosafety cabinet (class IIB) in a Biolevel III laboratory (e.g., in the Laboratory Building of Alberta Agriculture and Rural Development). Final content of the homogenate is about 2.5 and 5 grams (equivalent of fresh tissue) in TAD-tissue mixture in a low- and high-dose group, respectively. The mixture is then placed into a screw-capped, safety-coated glass bottle. Anaerobic digestion starts in an incubator with a temperature setting of 55° C. and pH 8 with specific controls (see Tab. 1 for study design).

TABLE 1
Experimental Design

| | Experiments | | | | Controls | | | |
|---|---|---|---|---|---|---|---|---|
| | | | | | | | IC (BSE brain and | |
| | N (normal bovine | | B (BSE bovine | | | | inactivated | |
| TAD-Tissue | brain) | | brain) | | DC (without brain) | | digest mixture) | |
| Mixture | N-low | N-high | B-low | B-high | DC-1 | DC-2 | IC-1 | IC-2 |
| Brain tissue | 2.5 | 5.0 | 2.5 | 5.0 | — | — | 2.5 | 5.0 |
| containing BSE | | | | | | | | |
| (grain) | | | | | | | | |
| Anaerobic | Same amount in each group (<250 mL) | | | | | | | |
| Digestate | | | | | | | | |
| Cellulose* | | 1 | | 1 | | 1 | | |
| (gram) | | | | | | | | |

| | |
|---|---|
| Incubation @ | 55° C. |

*Cellulose is added to the digestion mixture as a carbon-rich material to provide extra carbohydrate and may boost digestive activity of the anaerobic bacteria.

Inactivated digestate control (IC) is designed to check whether there is degradation of BSE (B) in the silent digestion mixture without activity of live bacteria. Additional control group (N) includes normal bovine brain homogenate containing cellular prion. This allows checking elimination rate of cellular prion during the digestion process. A correlation between the cellular and BSE prion predicts relative elimination rate of BSE prion during TAD process.

A similar experiment is also designed for TAD digesters containing bovine brain tissue and other types of tissue mixtures in comparison with bovine brain alone.

Biogas production and composition is monitored with a pressure transducer and gas chromatography. The time course of BSE prion decontamination is assessed at different time points from Day 0 to 120. At each time point, total protein from samples is extracted, concentrated and purified using established methods, and subjected to analyses using SDS-PAGE, Western blotting (WB, Schaller et al., 1999; Stack, 2004) with a panel of specific monoclonal anti-prion antibodies recognizing different epitopes. Reduction of the BSE prion in post-digest samples is compared with a series of 10-fold dilutions of the same batch of BSE brain homogenate and the sample taken at time zero. The WB image is analyzed using a densitometry to semi-quantify the reduction of the BSE prion at different times and with different tissue mixtures. For all positive samples detected by WB, the samples are subjected to proteinase-K digestion to examine whether resistance of BSE, prion has been altered during the TAD process.

Kinetics of BSE elimination in TAD is assessed using an equivalent amount of bovine brain homogenate containing cellular prion (PrP^{c}) as control. The rates of destruction of the bovine PrP^{c} and of the BSE priori are compared at different time points during the digestion process. A series of elimination percentiles of BSE at sequential time points provide relative kinetics of BSE destruction during the process.

### Example 3

### In Vitro Cyclic Amplification Misfolding Protein (iCAMP) Assay with High Sensitivity for Assessing the Completion of BSE Prion Destruction

Abnormal isoform of prion proteins (e.g., PrP^{sc}) retain infectivity even after undergoing routine sterilization processes. A sensitive method to detect the infectivity is a bioassay. However, the result of such bioassay can only be obtained after several hundred days. Hence, cyclic amplification of misfolding protein (CAMP) provides an attractive alternative in which PrP^{sc} can be amplified in vitro for assessing prion inactivation. Since three rounds of CAMP require only about 6 days, CAMP is much faster than the traditional bioassay.

An in vitro cyclic amplification mis-folding protein (iCAMP) method is developed herein for assessing the completion of BSE prion decontamination in TAD. Briefly, a 10% (w/v) homogenate of normal bovine brain and bovine brain with BSE is prepared in a conversion buffer. Specifically, iCAMP is set up with a volume of 50 μL containing different amounts of BSE prion (0.0001 to 1 g of the tissue equivalent) and a comparable amount of 10% (w/v) normal brain homogenate substrate. Amplification is conducted using a programmable sonicator with microplate horn (e.g., a Misonix S-3000 model) at 37° C. Amplification parameters are optimized using the following conditions: cycles: 40 to 150; power-on: 90 to 240 W; pulse-on time: 5 to 20 seconds; and interval: 30 to 60 minutes. Results of iCAMP are confirmed with WB (Western Blot) and PK digestion.

In the assessment strategy, if no BSE prion is detectable in TAD post-digest samples by WB, the sample is subjected to amplification using iCAMP. Purified post-digest samples is used as the “seed,” with 10% (w/v) bovine brain homogenate containing PrP^{c} as the substrate for iCAMP amplification. A serial dilution of brain homogenate containing BSE serves as a positive control. If a single motif of a mis-folded BSE prion protein still exists, the quantity of misfolding BSE prion is exponentially augmented by iCAMP. The sensitivity of iCAMP enables detection of a single motif of BSE prion protein (see Mahayana et al., Brioche Biophysics Rees Common 348: 758-762, 2006). If residual BSE is not detectable after 150 cycles, it indicates that BSE has been eradicated completely by the TAD process. iCAMP enables quick and efficient screening for a potential residual of BSE prion in post-digest samples, thus saving time and money that would otherwise be spent in animal-based bioassay.

Intracerebral inoculation of prions into mice or hamsters is a typical bioassay for assessing the infectivity of PrP^{sc} (Scott et al., Arch. Viral. (Suppl) 16: 113-124, 2000). Bioassay of BSE decontamination is conducted on those samples verified by iCAMP as “not detectable” using the transgenic mouse model. Transgenic (Tg) mice over-expressing full-length bovine PrP (Tg BoPrP) or inbred transgenic mouse is used for this purpose because of their susceptibility to BSE infection (Scott et al., Proc. Natl. Acad. Sci. USA 94: 14279-14284, 1997; Scott et al., J. Virol. 79: 5259-5271, 2005). Specifically, about 50 μL of filtrate-sterile iCAMP-negative sample is inoculated into mouse brain via a trephine of the skull under sterile conditions. Observation continues for 250 days or until clinical signs are developed. Some of the low-grade positive samples detected by WB, and WB negative/iCAMP positive samples is also subjected to mouse bioassay (FIG. 3, strategy of assessment). These assays enable determination of whether the infectivity of BSE prion has been eliminated or altered in TAD process post-digestively. Brain samples are taken for immunohistochemistry confirmation of disinfection of BSE using specific antibodies (Andréoletti, PrP^{sc} immunohistochemistry. In Techniques in Prion Research, Edited by Lehmann S and Grassi J, p 82, Birkhauser Verlag, Basel, Switzerland, 2004).

### Example 4

### Mechanisms of BSE Prion Disinfection in TAD

Complete decontamination of infectivity of BSE priori in TAD is expected to result from either entire degradation of or substantial structural and conformational changes to BSE prion proteins (Paramithiotis et al., 2003; Brown, 2003; Alexopoulos et al., 2007). These changes are investigated further using conformational assays and state-of-the-art mass spectrometry (Moroncini et al., 2006; Domon and Aehersold, 2006).

Mass spectrometry (MS) can determine peptide covalent structures and their modifications. Proteins from the post-digest samples are isolated, fractionated and digested to the peptides (Lo et al., 2007; Reiz et al., 2007a). A shotgun and/or comparative pattern analysis is used in MS analysis. Relative quantification of proteomic changes of any two comparative samples, such as digested and undigested ones, are carried out using differential stable isotope labeling of the peptides in the two samples followed by liquid chromatography MS (LC-MS) analysis (Ji et al., 2005a.b.c). This method is selective to detect and quantify only the proteins with abundance and/or sequence alternations in the two samples. Recent research has shown that various prion constructs including mis-folded prion aggregates can be digested sufficiently with or without trypsin, and 100% sequence coverage was obtained using the microwave-assisted acid hydrolysis (MAAH) (thong et al., 2004 and 2005; Wang et al., 2007: Reiz et al., 2007b).

To determine if BSE prion is degraded by TAD, structural alternation from amino acid modification and/or conformational change are probed by using MAAH, isotope labeling, LC-MS and/or MS/MS. If BSE prion is degraded by TAD, the resulting peptides can be identified by LC-MS/MS, which is useful in determining the potential protease(s) involved in cleaving the specific amino acid site(s).

Thermophilic anaerobic bacteria and their proteases play a significant role in destruction of BSE prions. A number of anaerobic bacterial species in the TAD digester containing BSE prion are identified with real time-PCR based genotyping of 16S ribosomal RNA gene (Ovreas et al., 1997). Functional analysis of proteolytic activities within the supernatant of the TAD-BSE mixture and/or of the bacterial isolates is carried out using the azocoll assay (Chavira Jr. et al., 1984; Müller-Hellwig et al., 2006). All these analyses facilitate the understanding of the mechanism(s) of BSE prion destruction, which may lead to the optimization of BSE decontamination strategy and potential drug discovery for prion-associated disorders.

### Example 5

### Using Protein-Enriched and Decontaminated BSE Prion-Containing Materials as Feedstock to Increase the Fuel Value of Biogas

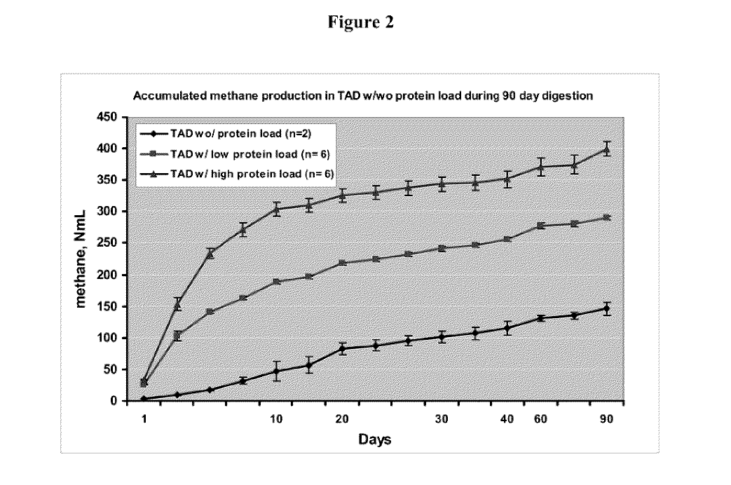

Preliminary results demonstrated the protein-load dependent-increase of biogas production (CO₂ plus CH₄) in the pilot study on scrapie inactivation (see Example 1). Accumulated methane in TAD containing high- and low-doses of scrapie and control brain tissue was about 2.75- and 1.70-folds higher respectively than that in TAD control without proteins during a course of digestion (FIG. 2).

In this experiment, biogas production profiles from TAD digesters containing RSE brain alone and BSE brain tissue mixed with other types of the tissues defined as SRM are compared. If the biogas profiles do not show differences, it indicates that anaerobic microbes treat different sources of tissue-derived proteins in a similar way. The comparative results of WB provides further evidence of whether decontamination of BSE priori is compromised by mixing the BSE brain tissue with other types of SRM tissues in TAD digester. It has been suggested that increased levels of ammonia due to protein/amino acid enrichment in the digestate inhibits TAD (Sung and Liu, 2003; Hartmann et al., 2005). In order to mitigate this effect (if any), the amount of protein load as feedstock in TAD can be optimized using existing computerized pilot plan and in the batch digester, respectively.

To further improve the system, ammonia in the biogas can be stripped during the TAD process. For example, ammonia can be captured by any ammonia-sorption materials (such as those described in US-2008-0047313-A1, incorporated by reference), which will turn ammonia (NH₃) into (NH₄)₂SO₄ or other compounds. The captured ammonia (such as (NH₄)₂SO₄) can be integrated into TAD effluent and then further processed to produce biofertilizer. This integrated technology will not only ensure productivity of the TAD process and high efficiency of BSE prion destruction, but will also increase biogas fuel value and market value of TAD effluents as a biofertilizer.

### Example 6

### Inactivation of Viruses Using Thermophilic Anaerobic Digestion

This example provides evidence that the thermophilic anaerobic digestion (TAD) process is capable of inactivating a model virus and its infectivity. The example also provides data concerning the dose- and time-dependent inactivation of TAD on the model virus. Furthermore, the example provides a platform to investigate the specific component(s) of TAD (e.g., enzyme, VFA, temperature, pH) that plays a role in viral disinfection.

The model virus used in the study is the Avian Herpesvirus (ATCC strain N-71851), a DNA virus. This virus causes outbreaks of infectious avian laryngotracheitis (ILT) and death of chicken. Susceptible cell line used in the study is LMH (ATCC CRL-2117), a hepatocellular carcinoma epithelial cell line. Infection of the LMH cell culture in vitro by the avian herpesvirus induces cytopathic effects (CPE, or cell death).

According to the study design, concentrated infectious viral stock was prepared by incubating ILT virus-infected LMH cell culture at 37° C. and under 5% CO₂. The resulting concentrated infectious viral stock was mixed with TAD filtrate, which was obtained by centrifuging a TAD digestate (55° C. anaerobic digestion), and filtering the supernatant through a 0.45 μm and a 0.22 μm filter, respectively. The mixture was allowed to be incubated at 37° C. for varied times (see below).

After incubation, a fixed amount of an aliquot of the mixture was applied to a monolayer of LMH cells grown on cover slips. The cells were then incubated at 37° C. for about 24-72 hrs, and the results examined under the microscope.

The results showed that a mere 30-minute pre-incubation of the ILTV stock with the TAD (thermophillic anaerobic digestion) sludge (centrifuged at about 10,000×g and filtered through 0.45 and 0.22 μm filters, either with or without neutralizing pH (original pH˜8.0)) aborted the appearance of CPE in the cultured LMH cells. This result indicates that some molecules in the filtrate of the TAD inhibited or inactivated ILTV, since the titrate was devoid of any live bacteria or virus after the double filtration.

The dose-dependent viral inactivation by TAD filtrate after 30-mm, pre-incubation was also measured. The results show that the tissue culture infection dose (TCID₅₀) for ILTV was 10⁸ dilution of stock virus. Wide-spread CPE occurred at 2 days at 1:1 ratio of ILTV stock:TAD filtrate. Moderate CPE occurred at 4 days at 1:4 ratio of ILTV stock:TAD filtrate. In contrast, no CPE occurred at 1:10, 1:20, or 1:100 ratio of ILTV stock:TAD filtrate. The results were summarized in the table below.

TABLE 2
Dose-dependent viral inactivation

| | Day 1 | Day 2 | Day 3 | Day 4 |
|---|---|---|---|---|
| Dose | (PS infect) | (PS infect) | (PS infect) | (PS infect) |
| 1 part virus/1 part TADF | V− | V+; CPE 25% | V+; CPE 50% | V+; CPE 75% |
| 1 part virus/2 parts TADF | V− | V+; CPE 25% | V+; CPE 50% | V+; CPE 75% |
| 1 part virus/5 parts TADF | V− | V− | V− | V+; CPE 25% |
| 1 part virus/10 parts TADF | V− | V− | V− | V−; No CPE |
| 1 part virus/100 parts TADF | V− | V− | V− | V−; No CPE |
| 1 part virus/1 part PBS | V+ | V+; CPE 25% | V+; CPE 50% | V+; CPE > 90% |

| | | |
|---|---|---|
| 1 part PBS/1 part TADF | V− | with good cell monolayer (no viral ctrl) |

* Detectable TCID₅ [symbol] , was 1 [symbol] 10⁻⁸ [symbol]
indicates data missing or illegible when filed

Time-dependent viral inactivation by TAD filtrate:ILTV stock at 1:1 ratio were also investigated. It was found that wide-spread CPE occurred in inoculated culture at 2 days after incubation of viral stock with TADF for 0, 10, 30 minutes at 37° C. Moderate CPE occurred in inoculated culture at 3 days after incubation of viral stock with TADF for 60 minutes at 37° C. Minimal CPE occurred in inoculated culture at 3 days after incubation of viral stock with TADF for 120 minutes at 37° C. The results were summarized in the table below.

TABLE 3
Time-dependent viral inactivation

| | Day 1 | Day 2 | Day 3 | Day 4 |
|---|---|---|---|---|
| Time | (PS infect) | (PS infect) | (PS infect) | (PS infect) |
| 0 min. | V−; CPE — | V−; CPE 25% | V+; CPE 50% | V+; CPE 75% |
| 10 min. | V−, CPE — | V−; CPE 25% | V+; CPE 50% | V+; CPE 75% |
| 20 min. | V−. CPE — | V?; CPE < 25% | V+; CPE 25% | V+; CPE 75% |
| 60 min. | V−; CPE — | V−; CPE — | V+: CPE 25% | V+; CPE 50% |
| 120 min. | V−; CPE — | V−; CPE — | V+; CPE < 25% | V+; CPE 25% |
| 120 min. ( PBS + virus) | V−; CPE — | V+; CPE 25% | V+; CPE 50% | V+; CPE 75% |

* ILTV: AD filtrate = 1:1

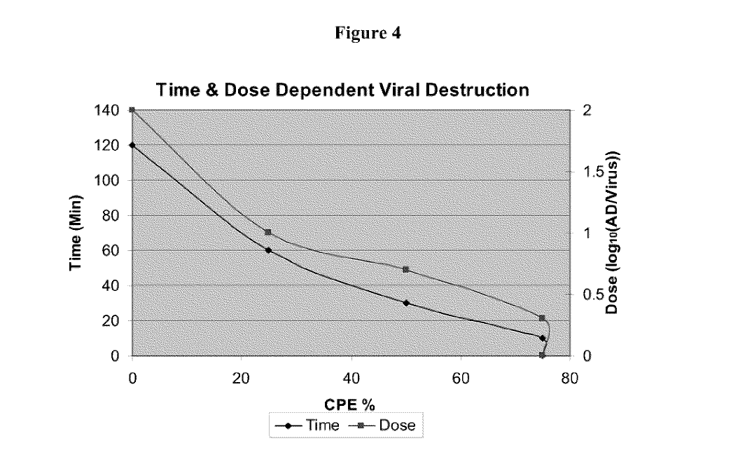

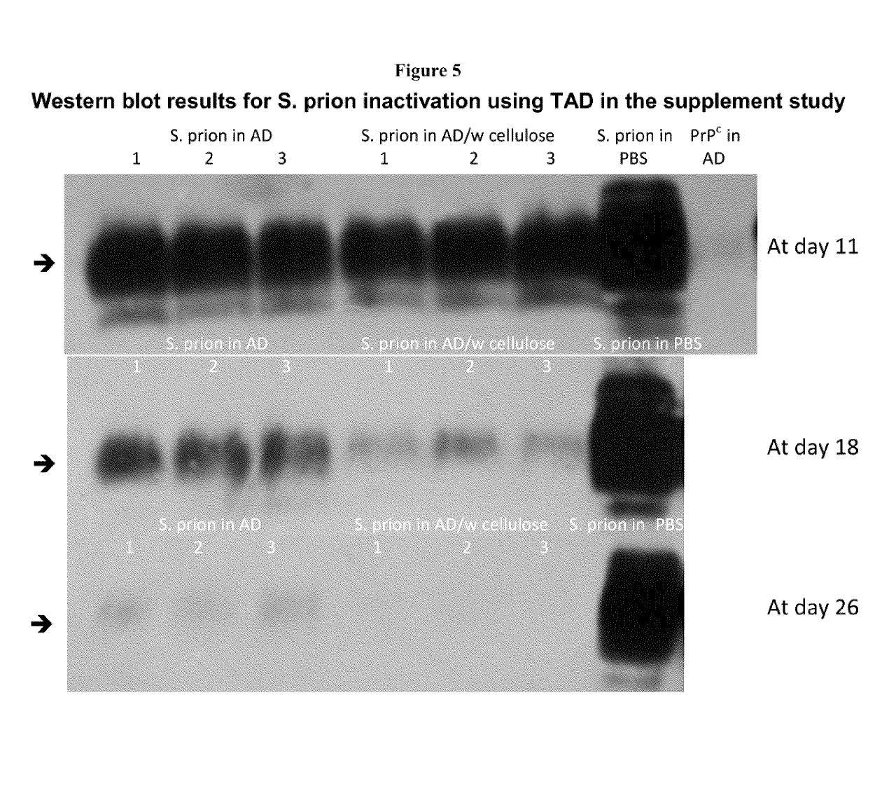

Results in Tables 2 and 3 are summarized in FIG. 4.

The experiments described in this example provide evidence that TAD filtrate alone (without anaerobic bacteria) can eliminate the infectivity of ILT virus in a dose- and time-dependent manner, when the infectious viral stock was pre-incubated with the filtrate. Although proteases or other bioactive enzymes in TAD filtrate do not seem to be major attributing factors to viral inactivation, volatile Fatty acid (VFA) at given concentration (e.g., >250 ppm) might play a role in viral inactivation.

Although the experiments used ILT virus, other viruses, especially other DNA viruses in the same family (including human viruses) can also be effectively destroyed in TAD process described herein. While not wishing to be bound by any particular theory, viral destruction may be a result of a synergistic effect between small metabolic molecules and complex anaerobic bacterial colonies in the TAD digestion system.

The exact identity of the small molecules critical for viral disinfection may be determined using any art-recognized methods, such as GS-MASS or HPLC-MASS, and nucleic acid testing.

### Example 7

### Removal of Infectivity of Infectious Laryngotracheitis Virus (ILTV) Using Thermophilic Anaerobic Digestion (TAD) Process

Infectious laryngotracheitis (ILT) is an upper-respiratory disease of poultry caused by a herpesvirus. It is a provincially reportable disease in Alberta, Canada. Because of its endemic nature, it is economically important to the provincial poultry industry. In areas of intense poultry production and during disease outbreaks, the virus causes significant loss of the birds and reduction in egg production.

The virus can survive in tracheal tissues of a bird up to 44 hours post mortem. Although ILT virus (ILTV) can be inactivated by organic solvents and high temperature (55° C. and above), the TAD process described herein provides a more cost-effective and environmentally responsible way to destroy this virus.

In this experiment, ILTV was successfully cultured in specific pathogen-free chicken embryos and an avian continuous cell line (chicken lung cell). The cells are highly susceptible to the virus, and exhibit characteristic cytopathic effects (CPE) 3 to 4 days post infection. The ILTV infected cells can readily be identified directly under microscope or using an indirect fluorescent test (IFAT).

In the first set of experiments, an equal volume of ILTV (challenge dose of 100,000 TCID 50) and the filtrate from active TAD (TAD-f) digestate (collected from the Integrated Manure Utilization System (IMUS™) demonstration plant, Vegreville) (TAD-f) were mixed and incubated at 37° C. for different periods of time (10, 30, 60 and 120 min.) before inoculation into the tissue culture cells. In the second set of experiments, TAD-f was mixed with 1 volume of virus suspension at different ratio of digestate vs. virus (1:1, 25:1, and 100:1) and incubated for 60 minutes before inoculation into the tissue culture cells. The control used for comparison was an untreated virus suspension with identical infectious dose inoculated into the cell line. The CPE of the cell cultures were scored after 3 to 4 days. The different incubation times and concentrations of TAD-f used were converted into log 10 and plotted against the percentages of CPE observed (data not shown).

We observed that, after an incubation period oft hours (120 min.), and similarly using the ratio of 100 times of TAD-f to 1 volume of virus suspension, the ILTV CRE has been eliminated, indicating that the infectivity of ILTV was removed completely. The percentages of CPE of ILTV were inversely proportional to the incubation time and amount of TAD-f added.

We have successfully demonstrated here a simple, inexpensive, and environmentally friendly TAD technology for disinfection of ILTV. In addition, the thermophilic anaerobic digestion system has been proven to generate renewable energy via biogas and reduce green-house gas emissions and the foot-print of agri-biowaste in the feedlot practice. Viral removal by TAD provides another environmentally friendly alternative to the poultry industry for controlling spread of ILT, and management of agri-biowaste.

### Example 8

### Evaluation of Pathogen in Biowaste and Digestate

There are many different types of waste products that are used for anaerobic digestion, however, biowaste that contains manure has a high density of coliform bacteria (1-6). The coliform bacteria can include pathogens associated with human illness, such as Salmonella and other zoonotic pathogens such as Campylobacter and Listeria (7-10). Generally, methods used to denote contamination in waste use indicator organisms like fecal coliform bacteria. For water, detection and enumeration of this group of organisms are used to determine the suitability of water for domestic and industrial use (11). In the United States, sludge from wastewater treatment plants must fulfill the density requirements from the US Environmental Protection Agency (USEPA) for fecal coliform as an indicator or Salmonella as a pathogen (12).

In the discussion presented by Pell (13) on pathogenic microbes in manure, there is mention that in the past, most environmental concerns about biowaste management have focused on nutrient overload, water quality or odor problems. There are no regulations concerning pathogens in biowaste that are used for anaerobic digestion. With an emerging biogas industry in Alberta, large amounts of effluent from anaerobic digesters will be produced. There is a lack of information as to whether pathogens are present in anaerobic digester effluent and if present, whether they will pose a threat to public, animal and plant health. We have found no information on regulations for handling effluent from anaerobic digesters for Alberta, although there is information on wastewater systems (14). Alberta Agriculture and Rural Development guidelines mention that land application of digestate is under the Agricultural Operations Practices Act and Regulations as it applies to manure (15). The Canadian Council for the Ministers of the Environment (CCME), in their guidelines for organism content in compost containing only yard waste, mention that fecal coliform of fecal origin should be <11000 Most Probable Number (MPN)/g of Total Solids (TS) calculated on a dry weight basis and Salmonella <3 MPN/4 g TS (16) and compost containing other feedstock should contain fecal coliform at <1000 MPN/g TS or Salmonella, <3 MPN/4 g TS. The compost with other feedstock must be exposed to 55° C. or higher for a specified time depending on the type of compost.

The USEPA have imposed regulations under Title 40 of the Code of Federal Regulations (CFR), Part 503 to control the use and disposal of biosolids (17). Biosolids are defined as the recyclable organic solid product produced during wastewater treatment processes. Part 503 of the rule gives the requirements for the use of biosolids in order to prevent contamination to the public and the environment. One requirement is for the control of pathogens or disease-causing organisms and the reduction of vector attraction to the biosolids. Pathogens can be bacteria, viruses and parasites and vectors include rodents, flies, mosquitoes and disease-carrying and transferring organisms. The rules described in Part 503 ensure that pathogen levels are safe for the biosolids to be land applied or surface disposed. The criteria for biosolid Class A are the same as the CCME guidelines for compost with other feedstock, with fecal coliform <1000 MPN/g TS or Salmonella <3 MPN/4 g TS. A biosolid is considered Class B if pathogens are reduced to levels that do not pose a risk to the public and environment. Measures must be taken to prevent crop harvesting, animal grazing and public access to areas where Class B biosolid have been applied until the area is considered safe. The Class B biosolid requirements are that fecal coliform must be <2·10⁶ MPN/g TS. For this biosolid, the fecal coliform is used as an indicator of average density of bacterial and viral pathogens.

We conducted a small-scale study on undigested biowaste and effluent after anaerobic digestion of biowaste using the USEPA microbiology testing methods for fecal coliform (18) and Salmonella (19) for biosolids and used the results to assess local biowaste samples. Due to time and resource limitations at the time of experiment, only selected analyses were performed on chosen biowaste samples.

### Objectives

to assess the levels of fecal coliform used as a contamination indicator and Salmonella used as pathogen indicator for selected biowaste samples
to evaluate reduction of fecal coliform and Salmonella using thermophilic anaerobic digestion processes

The results from this study provide preliminary data for development of guidelines for handling and utilizing biowaste.

### Biowaste and Sample Collection

All samples were collected into sterile plastic bags or bottles and tested within 2-3 hours after collection, unless otherwise stated. All samples were collected specifically for this study except sample 1.4, which was collected and stored at ARC, Vegreville, Alberta. This sample was being used in the ARC fully automated anaerobic digestion system ARC Pilot Plant (referred to as ARC Pilot Plant from here on) at the time of this study. The digestion system operated at 55° C. All dairy and chicken manure samples were collected from the same farm in the winter months. The farm was chosen because of its close proximity to the testing laboratory, allowing valid testing of fecal coliform and Salmonella within the required time frame for the USEPA microbiological testing methods.

The following samples were tested in this study:
1.1 Dairy manure taken from within dairy cows. Three dairy manure samples collected on two occasions from 5 dairy cows. Sample 1 was a manure mixture from cows 1 and 2, and Sample 2 was a mixture from cows 3 and 4. Sample 3 was from cow 5. One sample was tested for Salmonella only.
1.2 Dairy manure from one cow that was collected from the barn and tested for Salmonella only.
1.3 Dairy manure collected from the general barn area. Some of the freshly collected manure was taken to the Edmonton ARC laboratory. The remainder of the manure was transported to Vegreville and digested in the ARC Pilot Plant. At this time the digester was running dairy manure at 55° C. The freshly collected dairy manure was fed into the digester over ID days. The last feeding of manure was 15 hours before the sample was taken for analysis.
1.4 Dairy manure that was used routinely for TAD digestion at the ARC Pilot Plant. The dairy manure was collected from the same farm as samples 1.1 to 1.3 and stored for 2 months at 4° C. The stored sample and a random sample from the digester hopper were tested. The dairy manure from the hopper was diluted in the laboratory and left at 22° C. for 1 hour. A post-digested sample From the dairy manure was collected and tested.
1.5 Chicken manure, collected from chicken cages in the barn.
1.6 Chicken manure, collected from the general barn area and included straw bedding.
1.7 Household kitchen waste, mostly vegetable and fruit waste collected daily over a 7-day period and held at 4-6° C. until testing.
1.8 Broken eggs, including shell, collected at a grocery retail store that was close to the testing laboratory.
1.9 Wet distillers grain from an ethanol production plant, collected in barrels and stored at −20° C. until testing in the ARC Pilot Plant. This sample was collected for use in the ARC Pilot Plant and was chosen for pathogen analysis because it was a non-manure based biowaste. A diluted sample with 8% TS was taken for fecal coliform and Salmonella testing.

### Testing Methods

All dehydrated culture media were purchased from Neogen (Ml, USA) and testing, was carried out in a Biolevel II lab. A 5-tube MPN method was used as described in the USEPA methods to derive population estimates for the fecal conform and Salmonella.

### Total Solid Measurements of Biowaste

Total solid analysis was done for biowaste using a forced-air oven-drying method at 70° C. for 48 hours. The method assumes only water is removed. The results are reported as a percent of the sample's wet weight.

### Testing for Fecal Coliform

The biowaste and anaerobic digester effluent were evaluated for fecal conform using the USEPA Method 1680 (17). Briefly, the method uses a MPN procedure to derive a population estimate for fecal conform bacteria, Lauryl-Tryptose broth and EC culture specific media and elevated temperature to isolate and enumerate fecal coliform organisms. The basis for the test is that fecal coliform bacteria, including Escherichia coil (E. coli), are commonly found in the feces of humans and other warm-blooded animals.

These bacteria indicate the potential presence of other bacterial and viral pathogens. Total solids determination was done on the biowaste samples and used to calculate and report fecal coliform as MPN/g dry weight.

### Testing for Salmonella sp.

The biowaste and anaerobic digester effluent were evaluated for Salmonella using the USEPA Method 1682 (18). Briefly, the method is for the detection and enumeration of Salmonella by enrichment with tryptic soy broth and selection with modified semisolid Rappaport-Vassiliadis medium. Presumptive identification was done using xylose-lysine desoxycholate agar and confirmation was done using lysine-iron agar, triple sugar iron agar and urea broth. Serological testing was done. Total solids were determined on a representative biowaste sample and used to calculate Salmonella density as MPN per 4 g dry weight.

### Quality Control

Milorganite (CAS 8049-99-8, Milwaukee Metropolitan Sewerage District, UNGRO Corp. ON), a heat-dried Class A biosolid proven by USEPA was used and spiked with appropriate control bacteria. E. coli (ATCC#25922) was used as the positive control for the fecal coliform test and negative control for the Salmonella test. Salmonella typhimurium (ATCC#14028) was used as the positive control for the Salmonella test.

Enterobacter aerogenes (ATCC#13048) and Pseudomonas (ATCC#27853) were used as negative controls for the fecal coliform test.

### Results and Discussion

The table below gives the total solid, fecal coliform and Salmonella MPN for the biowaste samples.

Summary of microbiology testing results of selected biowaste samples

| | | Total solids | Fecal coliform | Salmonella |
|---|---|---|---|---|
| | Samples | (% of wet weight) | (MPN/g TS) | (MPN/4 g TS) |
| 1.1 | Dairy manure taken from within dairy cows | | | |
| | Sample 1 | 13 | 5.6 [symbol] 10⁶ | <0.18 |
| | Sample 2 | 15 | 1.1 [symbol] 10⁷ | <0.18 |
| | Sample 3 | 14^{a} | Not done | <0.18 |

| | |
|---|---|
| 1.2 | Dairy manure from general barn area |

| | | |
|---|---|---|
| 14^{a} | Not done | <0.18 |

| | |
|---|---|
| 1.3 | Dairy manure from general barn area |

| | | |
|---|---|---|
| 15 | 1.1 [symbol] 10⁷ | 4.0 × 10 [symbol] |

Anaerobic digestion effluent of dairy manure after 15 hrs digestion

| | | |
|---|---|---|
| 10 | <0.18 | <0.18 |

| | |
|---|---|
| 1.4 | Dairy manure used at ARC Pilot Plant |
| | Dairy manure stored for 2 months at 4° C. |

| | | |
|---|---|---|
| 14 | 8.8 [symbol] 10 [symbol] | <0.18 |

Dairy collected from ARC Pilot Plant hopper before anaerobic digestion

| | | |
|---|---|---|
| 10 | 1.8 [symbol] 10 [symbol] | 2.1 [symbol] 10 [symbol] |

Anaerobic digestion effluent of dairy manure after 15 hours hydraulic retention time

| | | |
|---|---|---|
| 9 | <0.18 | <0.18 |

| | |
|---|---|
| 1.5 | Chicken manure from cages |

| | | |
|---|---|---|
| 37 | 4.3 [symbol] 10⁶ | <0.18 |

| | |
|---|---|
| 1.6 | Chicken manure from general barn area with straw bedding |

| | | |
|---|---|---|
| 78 | 2.1 [symbol] 10⁶ | <0.18 |

| | |
|---|---|
| 1.7 | Household kitchen waste |

| | | |
|---|---|---|
| Not done | No growth | No growth |

| | |
|---|---|
| 1.8 | Broken eggs |

| | | |
|---|---|---|
| Not done | No growth | No growth |

| | |
|---|---|
| 1.9 | Wet distillers grains |

| | | |
|---|---|---|
| 8 | <0.18 | <0.18 |

^{a}Estimated TS values [symbol]
indicates data missing or illegible when filed

Dairy manure samples from the same facility were tested in this study. The samples were from the general barn area and taken from within cows. When tested, the density of fecal coliform that was found in all samples ranged from 8.8×10⁴ MPN/g TS to 1.1×10⁷ MPN/g TS. Salmonella, 4×10⁶ MPN/4 g TS, was found in one sample collected from the general barn area. Storage of the dairy manure at 4° C. for 2 months decreased the fecal coliform 2- to 3-log. In both cases where dairy manure was digested at 55° C. by TAD digested for 15 hours, the fecal coliform and Salmonella were decreased to below detection (<0.18 MPN/g TS for fecal coliform and <0.18 MPN/4 g TS for Salmonella).

The chicken manures, kitchen waste, eggs and wet distillers grain were not put through digestion. Both chicken manure samples had fecal coliform, 4.3×10⁶ and 2.1×10⁶ MPN/g TS. No Salmonella was detected. There were no fecal coliform and Salmonella in the kitchen waste, eggs and wet distillers grains.

This brief study showed that bacteria common to manures were detected in the dairy and chicken manure samples. According to the USEPA guidelines for a Class A biosolid, the fecal coliform density was above the accepted level in all manure samples, and for a Class B biosolid, the fecal coliform density was above the accepted level in the freshly collected manure samples. The increased fecal coliform levels indicate that pathogenic bacteria could be present in these samples. This was verified by the fact that one fresh dairy sample contained 4.0×10⁶ MPN/4 g TS and a random hopper sample from the ARC Pilot Plant contained 2.1×10⁶ MPN/4 g TS Salmonella. The sample was tested to contain below detection levels of both fecal coliform and Salmonella after anaerobic digestion at 55° C. for 15 hours.

Bendixen (20) looked at the animal and human pathogen reduction in Danish biogas plants. It was reported that pathogen survival was greatly reduced at thermophilic digestion temperatures (50° C. to 55° C.) but not at low and mesophilic temperatures (5° C. to 45° C.). Biogas plant construction, function and management need to be monitored in order to assure pathogen destruction and policies need to be in place to classify the digested effluent for proper disposal. The requirements in the USEPA standards (17) for sewage sludge use and disposal indicate that sewage sludge should be analyzed for enteric viruses and viable helminth ova. There are also requirements given for vector attraction reduction and reduction of volatile solids. As well, other pathogens should be investigated. For example, human norovirus strains have been found in livestock, indicating a route for zoonotic transmission (21). As well, policies have been made concerning plant pathogens that relate to anaerobic digestion facilities in Germany (22).

### SUMMARY

Using the USEPA Class A biosolids and CCME guideline for compost of <1000 MPN/g TS for fecal conform, all the freshly collected manures (dairy and chicken) were above the accepted level.
Using the USEPA Class B biosolids guidelines of <2·10⁶ MPN/g TS for fecal coliform, all the freshly collected manure samples (dairy and chicken) were above the accepted level.
For one fresh dairy manure, the Salmonella exceeded the USEPA Class A biosolids and CCME guideline for compost of <3 MPN/4 g TS.
Storage of dairy manure at 4° C. for 2 months decreased fecal coliform concentration.
Anaerobic digestion at 55° C. for 15 hours reduced fecal coliform and Salmonella to below detection levels. Fifteen hours of digestion in a continuous stirred tank reactor system appeared to be adequate for reduction.
Household kitchen waste, broken eggs and wet distillers grains contained either no fecal coliform and Salmonella or levels below detection using the MPN method.

### REFERENCES FOR EXAMPLE 8

1. Weaver R W, J A Entry and A Graves. 2005. “Numbers of fecal streptococci and Escherichia coli in fresh and dry cattle, horse, and sheep manure,” Can. J. Microbiol. 51: 847-851.
2. Poppe C, R J Irwin, S Messier, G G Finley and J Oggel. 1991. “The prevalence of Salmonella enterilidis and other Salmonella spp. among Canadian registered commercial chicken broiler flocks,” Epidemiol. Infect. 107: 201-2011.
3. Poppe C, R J Irwin, C M Forsberg, R C Clarke and J Oggel. 1991. “The prevalence of Salmonella enterilidis and other Salmonella spp. among Canadian registered commercial layer flocks.” Epidemiol. Infect. 106: 259-270.
4. Morgan J A, A E Hoet, T E Wittum, C M Monahan and J F Martin. 2008. “Reduction of pathogenic indicator organisms in dairy wastewater using an ecological treatment system.” J. Environ. Qual. 37:272-279.
5. Sullivan T J, J A Moore, D R Thomas, E Mallery, K U Snyder, M Wustenberg, J Wustenberg, S D Mackey and D L Moore. 2007. “Efficacy of vegetated buffers in preventing transport of fecal coliform bacteria from pasturelands.” 40(6): 958-965.
6. Khakhria R, D Woodward, W M Johnson and C Poppe. 1997. “Salmonella isolated from humans, animals and other sources in Canada, 1983-92.” Epidemiol. Infect. 119: 15-23.
7. Rodrigue D C, R V Tauxe and B Rowe. 1990. “International increase in Salmonella enterilidis: A new pandemic?” Epidemiol. Infra. 105: 21-27.
8. Pradhan A K, J S Van Kessel, J S Karns, D R Wolfgang, E Hovingh, K A Nelen, J M Smith, R H Whitlock, T Fyock, S Ladely, P J Fedorka-Cray and Y H Schukken. 2009. “Dynamics of endemic infectious diseases of animal and human importance on three dairy herds in the northeastern United States.” 92 (4): 1811-1825.
9. Talbot E A, E R Gagnon and J Greenblatt. 2006. “Common ground for the control of multidrug-resistant Salmonella in ground beef,” Clin. Infect. Dis. 42:1455-62, 2006.
10. Stralcy B A, Donaldson S C, Hedga N V, Sawant A A, Srinivasan V, Olivier S P. 2006. “Public health significance of antimicrobial-resistant gram-negative bacteria in raw tank milk,” Foodborne Pathog. Dis. 3(3):222-233, 2006.
11. Clesceri L S, A E Greenberg and A D Eaton (Eds), 1998. Part 9000, “Microbiological Examination,” in Standard Methods for the Examination of Water and Wastewater. 20th edition. pp. 9-1.
12. Iranpour R, H H J Cox. 2006. “Recurrence of fecal conforms and Salmonella species in biosolids following thermophilic anaerobic digestion,” Water Environ. Res. 78(9): 1005-1012.
13. Pell A N. 1997. “Manure and microbes: Public and animal health problem?” J. Daily Sci. 80: 2673-2681.
14. Alberta Environment. 2006, “Standards and guidelines for municipal waterworks, wastewater and storm drainage systems,” Pub. No. T/840. ISBN, 0-7785-4394-3. Alberta Environment, Edmonton.
15. 2008 Agriculture Operation Practices Act Reference Guide. 2008. Agriculture and Rural Development. Government of Alberta. Alberta Agriculture and Rural Development, Government of Alberta.
16. CCME (Canadian Council of Ministers of the Environment). 2005. Guidelines for compost quality. PN 1340. Winnipeg, Canada.
17. US EPA (United States Environmental Protection Agency). 2007. Title 40: “Protection of the Environment,” part 503, Standards the use or disposal of sewage sludge. US Environmental Protection Agency, Washington D.C.
18. US EPA (United States Environment Protection Agency), 2006. “Method 1680: Fecal coliforms in sewage sludge (Biosolids) by multiple-tube fermentation using Lauryl Tryptose Broth (LTB) and EC medium.” EPA-821-R-06-012. US Environment Protection Agency: Washington D.C.
19. US EPS (United States Environment Protection Agency). 2006. “Method 1682: Salmonella in sewage sludge (Biosolids) by modified semisolid Rappaport-Vassiliadis (MSRV) medium.” EPA-821-R-06-14. US Environment Protection Agency: Washington D.C.
20. Bendixen H J. “Safeguards against pathogens in Danish biogas plants.” 1994. Wat. Sci. Tech. 30(12): 171-180.
21. Mattison K, A Shukla, A Cook, F Pollari, R Friendship, D Kelton, S Bidawid and J M Farber. “Human Noroviruses in swine and cattle,” Emerg. Infect. Dis. 13(8): 1184-1188.
22. Ordinance on the “Utilization of Biowastes on Land used for Agricultural, Silvicultural and Horticultural Purposes.” 1998. Ordinance on Biowastes BioAbfV. Germany.

### Example 9

### Enhanced Prion Destruction Using Thermophilic Anaerobic Digestion (TAD) Process

Applicants demonstrate in this example that prion destruction is also enhanced by adding carbohydrate-based substrate (non-protein substrate) into the digester and keep a consortium of anaerobes in active status.

Applicants previously showed that, biogas profile (CH₄ and CO₂) in batch digestion reached a peak at day 8 to 11, and then quickly dropped to a baseline level without further addition of substrate into the digestion. This result indicates that most of the anaerobes were in the resting state after the leveling off occurred.

In this study, cellulose substrate was added periodically (about every 7 days) starting day 11 into one study group of TAD digestion with 10 ml of 40% scrapie brain tissue. As a control, another study group was similarly set up (TAD digestion with 10 ml of 40% scrapie brain tissue), but without the additional of additional cellulose substrates, as in the previous study. The study was carried on for 90 days. Sampling schedule was as follows: day 0, 6, 11, 18, 26, 40, 60 and 90. At the end of the study, the scrapie prion was extracted, purified, desalted, and concentrated for analysis using 12% SDS-PAGE and Western blot. Western blot images were semi-quantified using Alpha Innotech Image Analyzer (Multilmage II, Alpha Innotech, San Leandro, Calif.).

The results from the image analysis show the following:

1) In the control group of TAD with scrapie prion only (no added cellulose substrate), 2.2 log reduction of scrapie prion was achieved at day 26 comparing to the starting amount of scrapie prion in TAD at day 0, and the amount of scrapie prion spiked in phosphate buffer (PBS) at day 26, respectively. This result was the same as shown in the previous study.

2) In the group of TAD with scrapie prion and additional cellulose substrate, more than 3 logs of reduction of scrapie prion was achieved at day 26 comparing to the starting amount of scrapie prion in TAD at day 0, and the amount of scrapie spiked in PBS at day 26, respectively.

3) TAD only eliminated 0.8 logs of scrapie prion (from 12.18 to 11.38 logs of integrated density and area (IDA)) while and TAD with additional cellulose substrate (1 gram in 60 ml of TAD/scrapie prion mix) eliminated 1.37 logs of scrapie prion (from 12.15 to 10.78 logs of IDA) (p<0.001, student-t test), from day 11 to 18.

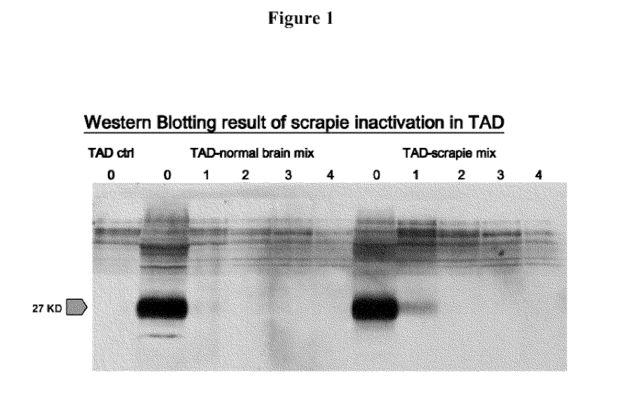

4) TAD eliminated 1.05 logs of scrapie prion (from 11.38 to 10.34 logs of IDA), while TAD with the second cycle of additional cellulose substrate eliminated scrapie prion to undetectable level in the current Western blot method, from day to 18 to 26. It is expected that more than 2 log further reduction could be achieved during this period after the second addition of cellulose substrate (FIG. 1. Western blot image showing the reduction of scrapie prion from day 11 to day 26).

5) A computational modeling is being carried out to predict destruction rate of scrapie prion using TAD process with and without addition of carbohydrate-based substrate. The modeling allows Applicants to avoid the limitation of detection sensitivity using the current available methods in the field of prion disease research and diagnostics.

In summary, the subject TAD technology can effectively destroy scrapie prion proteins in a time-dependent manner. Adding carbohydrate-based and non-protein containing substrates periodically into TAD process enhanced destruction capability. It is estimated that more than 3 logs of reduction of scrapie prion titers was obtained at day 26 in the group with additional carbohydrate-based (non-protein containing) substrates. Based on the experimental data, a computational modeling can be used to predict the time course of prion reduction in TAD process, and the time it takes to achieve substantially complete eradication of prion in SRM.

### GENERAL REFERENCES

Alexopoulos et al., PrP^{res}-specific antibody 4C2: crystal structure of its Fab fragment and docking calculations. P29, Abstract book, PrP Canada 2007, February 18-20, Calgary.
Andréoletti, “PrP^{sc} immunohistochemistry.” In Techniques in Prion Research, Edited by Lehmann S and Grassi J, p 82, Birkhauser Verlag, Basel, Switzerland, 2004.
Angelidaki et al., “Thermophilic anaerobic digestion of source-sorted organic fraction of household municipal solid waste,” Wafer Res. 40: 2621, 2006.
Angenent et al., “Methanogenic population dynamics during start-up of a full-scale anaerobic sequencing hatch reactor treating swine waste,” Water Res. 36: 4648, 2002.
Atarashi et al., “Ultrasensitive detection of scrapie prion protein using seeded conversion of recombinant prion protein,” Nat. Meth. 4: 645-50, 2007.
Belay et al., “Chronic Wasting Disease and potential transmission to human,” Emerging Infec Dis 10: 977-984, 2004.
Bragg, “Enhancements to Canada's Ruminant Feed Ban.” Presentation, September 2006.
Brown et al., Chemical disinfection of Creutzfeldt-Jakob disease virus. N. Eng. J. Med. 306: 1279, 1982.
Brown et al., Sodium hydroxide decontamination of Creutzfeldt-Jakob disease virus. N. Eng. J. Med. 310: 727, 1984.
Brown et al., “Newer data on the inactivation of scrapie virus or Creutzfeldt-Jakob disease virus in brain,” J. Infect. Dis 161: 467, 1986.
Brown Dr, “Conformational exposure: a new handle on prions,” Lancet 362: 929-30, 2003.
Canada Gazette. “Regulations Amending Certain Regulations Administered and Enforced by the Canadian Food Inspection Agency,” Canada Gazelle, Part 11, 140-14, SOR/2006-147, Jul. 12, 2006.
Castilla and Soto, “Cyclic Amplification of Prion Protein Misfolding,” Techniques in Prion Research, Edited by Lehmann S and Grassi 0.1, p 198, Birkhauser Verlag, Basel, Switzerland, 2004.
Castilla and Soto, “Detection of prions in blood,” Nat. Med. 11: 982-985, 2005a.
Castilla et al., “In vitro generation of infectious scrapie prions,” Cell 121: 195-206, 20050.
Caughey et al, “Cell-free formation of protease-resistant prion protein,” J. Virol. 71: 4107, 1997.
Chavira et al., “Assaying proteinase with azocoll,” Anal. Biochem. 136: 446-50, 1984.
Chatigny and Prusiner, “Biohazards and risk assessment of laboratory studies on the agents casing the spongiform encephalopathies.” In: Slow Transmissible Diseases at the Nervous System, Academic Press, New York, pp. 491, 1979.
CFIA publication. “Industrial Treatment of Specified Risk Materials: A Qualitative Risk Assessment of BSE Transmission and Spread to Domestic Ruminant.” (N26), Canadian Food Inspection Agency, July 2006.
Collinge, “Prion diseases of human and animals: their causes and molecular basis,” Annu. Rev. Neurosci. 24: 519, 2001.
Domon and Aebersold, “Mass spectrometry and protein analysis,” Science 312(5771): 212-7, 2006.
Gavala and Lyberatos, “Influences of anaerobic culture acclimation on the degradation kinetics of various substrates,” Biotech. Bioeng. 74: 181-95, 2001.
Gallert et al., “Effect of ammonia on the anaerobic degradation of protein by mesophilic and thermophilic biowaste population,” Appl. Microbiol. Biotechnol. 50: 495-501, 1998.
Facklam, “CFIA's Enhanced Feed Ban—To further protect the health of Canada's national cattle herd and acceleration of eradication of BSE,” Presentation, 2007.
Hartmann and Ahring, “A novel process configuration for anaerobic digestion of source-sorted household waster using hyper-thermophilic post-treatment,” Biotechnol. Bioeng. 90: 830, 2005.
Huang et al., “Evidence for degradation of abnormal prion protein in tissue from sheep with scrapie during compositing,” Canadian J. of Vet Res. 71: 34-40, 2007a.
Huang et al., In vitro degradation by microbes of abnormal prions in tissues of scrapie affected sheep. P24, Abstract Book, PrP Canada 2007b, February 18-20, Calgary.
Hui et al., “Alkaline serine protease produced by Streptomyces sp. degrades PrP^{sc},” Biochem. Biophys. Res. Commun. 321: 45-50, 2004.
Ji et al., “Identification and Quantification of Differentially Expressed Proteins in E-Cadherin Deficient SCC9 Cells and SCC9 Transfectants Expressing E-Cadherin by Dimethyl Isotope Labeling, LC-MALDI MS and MS/MS,” J. Proteome Res. 4: 1419-26, 2005a.
Ji and Li, “Quantitative Proteome Analysis Using Differential Stable Isotopic Labeling and Microbore LC-MALDI MS and MS/MS,” J. Proteome Res. 4: 734-42, 2005b.
Ji et al., “Differential Dimethyl Labeling of N-termini of Peptides after Guanidination for Proteome Analysis,” J. Proteome Res. 4: 2099-2108, 2005c.
Kim et al., “Comparative process stability and efficiency of anaerobic digestion: mesophilic vs thermophilic,” Water Res. 36: 4369, 2002.
Kirchmayr et al., “Prion protein-Detection in spiked anaerobic sludge and degradation experiments under anaerobic conditions,” ADSW 2005 Conference Proceedings Vol 1: 333-339, 2005.
Kirchmayr et al., “Prion protein: detection in spiked anaerobic sludge and degradation experiments under anaerobic conditions,” Water Science Technology, 53: 91-98, 2006.
Kocisko et al., “Scrapie infectivity correlates with converting activity, protease resistance, aggregation of scrapie-associated prion proteins in guanidine denaturation studies,” Nature. 370: 471, 1994.
Langeveld et al., “Enzymatic degradation of prion protein in brain stem from infected cattle and sheep,” J. Infect. Dis. 188: 1782-89, 2003.
Le et al., The losses from BSE in Canada. Presentation at the Canadian Agricultural Trade Policy Research Network. Toronto. Ontario, Feb. 11, 2006.
Lo et al., Optimization of the relative quantification of peptide using dimethylation-guanidination (2MEGA) and LC-MALDI-MS MS. P25, Abstract hook, PrP Canada 2007, February 18-20, Calgary.
McLeod et al., “Proteolytic inactivation of the bovine spongiform encephalopathy agent,” Biochem. Bioph. Res. Communi. 317: 1165-1170, 2004.
Murayama et al., “Protein misfolding cyclic amplification as a rapid test for assessment of prion inactivation,” Biochem. Biophys. Res. Commun. 348: 758-762, 2006.
Millson et al., “The physico-chemical nature of the scrapie agent,” Slow limas Diseases of Animals and Man, Edited by Kimberlin R H, North-Holland Publishing Company, pp 244-266, 1976.
Mitura and Di Pietro, “Canada's Beef Cattle Sector and the Impact of BSE on Farm Family Income: 2000-2003” Statistics Canada. Agriculture and Rural Working Paper Series. Cat No. 21-601-MIE-No 069, 2005
Moroncini et al., “Pathologic prion protein is specifically recognized in situ by a novel PrP conformational antibody,” Neurobiol. Dis. 23: 717-24, 2006.
Müller-Hellwig et al., “Biochemical evidence for the protelytic degradation of infectious prion protein PrP^{sc} in hamster brain homogenates by foodborne bacteria,” System Appl. Microbiol. 29: 165-171, 2006.
Ovreas et al., “Distribution of bacterioplankton in meromictic lake Sælenvannt, as determined by degenerate gradient gel electrophoresis of PCR amplified gene fragments coding for 16S rRNA,” Appl. Enviro. Microl., 63: 3367 73, 1997.
Paramithiotis et al., “A prion protein epitope selective for the pathologically misfolded conformation,” Nat. Med. 9: 893-9, 2003.
Pavlostathis and Giraldo-Gomez, “Kinetics of anaerobic treatment,” Wat. Sci. Tech. 24: 35-59, 1991.
Prusiner et al., “Thiocyanate and hydroxyl ions inactivate the scrapie agent,” Proc. Natl. Acad. Sci. USA 78: 4606, 1981.
Prusiner, “Prions,” Proc. Natl. Acad. Sci. USA 95: 13363-13383, 1998.
Reiz et al., Sequencing the prion protein using microwave assisted acid hydrolysis combined with liquid chromatography. P27, Abstract book, PrP Canada 2007a, February 18-20, Calgary.
Reiz et al., “Microwave-Assisted Acid Hydrolysis Combined with MALDI MS for Studying Prion Structure,” in Proceedings of the 55th ASMS Conference on Mass Spectrometry, Jun. 3-7, 2007b, Indianapolis, Ind.
Saborio et al., “Sensitive detection of pathological prion proteins by cyclic amplification of protein misfolding,” Nature 411: 810-813, 2001.
Schaller et al., “Validation of a Western immunoblotting procedure for bovine PrP^{sc} detection and its use as a rapid surveillance method for the diagnosis of bovine spongiform encephalopathy,” Acta Neuropathol. 98: 437-43, 1999.
Scherbel et al., “Degradation of scrapie associated prim protein by the gastrointestinal microbiota of cattle,” Vet Res. 37: 695-703, 2006.
Scott et al., “Identification of a prion protein epitope modulating transmission of bovine spongiform encephalopathy prions to transgenic mice,” Proc Natl Acad Sci USA 94: 14279-84, 1997.
Scott et al., “Transgenic models of prion disease,” Arch. Virol. (Suppl) 16: 113-24, 2000.
Scott et al., “Transmission barriers for bovine, ovine, and human prions in transgenic mice,” J. Virol. 79:5259-71, 2005.
Shigematsu et al., “Effect of dilution rate on structure of a mesophile acetate-degrading methanogenic community during continuing cultivation,” J. Biosci. Bioeng. 96: 547-58, 2003.
Stack, “Western immunoblotting techniques for the study of transmissible spongiform encephalopathies,” in Techniques in Prion Research, Edited by Lehmann S and Grassi J, p 97, Birkhauser Verlag, Basel, Switzerland, 2004.
Soto et al., “Pre-symptomatic detection of prions by cyclic amplification of protein misfolding,” FEBS Letters 579: 638-642, 2005.
Sung and Liu. “Ammonia inhibition on thermophilic anaerobic digestion,” Chemosphere 53: 43, 2003.
Suzuki et al., “Decomposition of extremely hard-to-degrade animal proteins by thermophilic bacteria,” J. Biosci. Bioeng. 102: 73-81, 2006.
Saa et al., “Presymptomatic detection of prions in blood,” Science 313: 92-94, 2006.
Tang et al., “Microbial community analysis of mesophilic anaerobic protein degradation process using bovine serum albumin-fed continues cultivation,” J. Biosci. Bioeng. 99: 150, 2005.
Taylor, “Inactivation of the unconventional agents of scrapie, bovine spongiform encephalopathy and CJD,” J. Hosp. Infect. 18: 141, 1991.
Taylor, “Inactivation of SE agents.” British Medical Bulletin 49: 810-821, 1993.
Taylor et al., “Inactivation of the 22A strain of scrapie agent by autoclaving in sodium hydroxide,” Vet. Microbiol. 58: 87-91, 1997.
Taylor, “Inactivation of transmissible degenerative encephalopathy agents,” Vet. J. 159: 10-17, 2000.
Taylor and Woodgate, “Rendering practices and inactivation of transmissible spongiform encephalopathy agents,” Rev. Sci. Tech. Off. Int. Epiz. 2003: 297-310, 2003.
Thackray al., “Proteinase K-sensitive disease-associated ovine prion protein revealed by conformation-dependent immunoassay,” Biochem. J. 401: 475-83, 2007.
Tsiroulnikov et al., “Hydrolysis of the amyloid prion protein and nonpathogenic meat and bone meal by anaerobic thermophilic prokaryotes and streptomyces subspecies,” J. Agri. Food Chem. 52: 6353-6360, 7004.
Wang et al., “Enzymatic degradation of a prion-like protein, Sup35 NM-His6,” Enzyme Micro. Tech. 36: 758-765, 2005.
Wang et al., “Proteome Profile of Cytosolic Component of Zebrafish Liver Generated by LC-ESI MS/MS Combined with Trypsin Digestion and Microwave-Assisted Acid Hydrolysis,” J. Proteome Res. 6: 263-72, 2007.
Yoshioka et al., “Characterization of a proteolytic enzyme derived from a Bacillus strain that effectively degrades prion protein,” J. Appl. Microbiol. 102: 509-15, 2007.
Zhong et al., “Protein Sequencing by Mass Analysis of Polypeptide-Ladders after Controlled Protein Hydrolysis,” Nature Biotechnology 22: 1291-96, 2004.
Zhong et al., “Microwave-Assisted Acid Hydrolysis of Proteins Combined with LC MALDI MS/MS for Protein identification,” J. Am. Soc. Mass Spectrum 16: 471-81, 2005.

### Example 10

### Antibiotic and Hormone Destruction Using Thermophilic Anaerobic Digestion (TAD) Process

Laboratory study using the lab-scale anaerobic digester with multiple samples (4 repeats in each group) was carried out using selected antibiotics and hormones with known dosages. Such antibiotics and hormones were spiked into the lab-scale digesters and digested for 4 weeks. The antibiotics and hormones were recovered by extraction and submitted to instrumental analysis.

Selected antibiotics and hormone used in the study include antibiotics and hormone commonly used in the feedlot practice in Northern America, including three antibiotics (Chlortetracycline, Tylosin and sulfamethazine) and one hormone (megestrol).

Advanced HPLC-DAD-ESI-MS system was used as analytic methods for analysis.

Detailed study design, groups and duration are listed in the table below:

### Study Design and Groups in Lab-Scale Studies

| | Antibiotics/ | Organic | | Sterile | Temperature |
|---|---|---|---|---|---|
| | hormones | Manure | TAD | water | (° C.) |
| Group 1 (exp) | Y | Y | Y | | 55 |
| Group 2 (ctrl) | Y | Y | | | 55 |
| Group 3 (ctrl) | Y | | | Y | 55 |
| Group 4 (ctrl) | Y | | | Y | 22 |

Duration of study: 4 weeks and samples were taken at day 2, 7, 14, 21 and 28 during the digestion process and submitted for instrumental analysis.

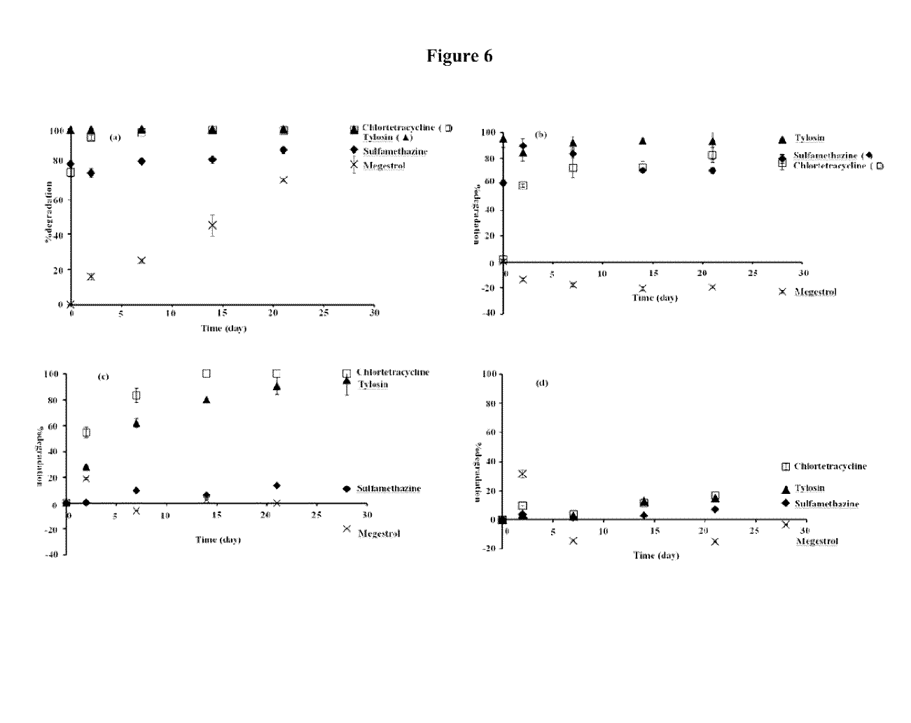

In comparison with the water control group at 22° C. (G4), the selected antibiotics, Chlortetracycline, Tylosin and sulfamethazine, were degraded with varied rates in other 3 groups (01, 2 and 3) setting at high temperature (55° C.). Chlortetracycline and Tylosin (>90%) were degraded rapidly (at day 2) in active TAD (G1) (FIG. 6a); Followed in fresh manure mixed with water control (G2) (60% chlortetracycline and 80% tylosin at day 2) (FIG. 6b), and then in water control (G3) (˜40%, chlortetracycline and ˜20% tylosin at day 2) (FIG. 6c). In General, =80% of chlortetracycline and tylosin were degraded by high temperature only within 3 weeks despite of liquid matrix. 100% degradation of two antibiotics was achieved by TAD process within one week.

In comparison to chlortetracycline and tylosin, sulfamethazine was degraded with less efficiency in TAD group (G1) and in manure mixed with water control (G2). 80 to 90% of degradation was observed at 4 weeks (FIGS. 6a and 6b). However, there was no noticeable degradation of sulfamethazine in water control with 55° C. setting (G3) (FIG. 6c).

In the water control group with low temperature (G4), there was no degradation of megestrol, and very limited degradation of all selected antibiotics (10 to 30%) (FIG. 6d).

A dynamic degradation of hormone (Megestrol) was only observed in active TAD group (G1). ˜80% of degradation of original amounts was achieved at 4 weeks of digestion process (FIG. 6a). There was no degradation of megestrol in other control groups.

The data here demonstrate that, about 80 to 100% of selected antibiotics and >50% megestrol can be degraded within two weeks of the TAD process. This degradation will also occur for the antibiotics and hormones with similar chemical structures.

High temperature alone could assist the degradation of some selected antibiotics. However, the degradation will not be completed without microbiological/biological process during anaerobic digestion. Hormones degradation and removal only occurred in TAD process.

### Example 11

### Biodegradation of Hormones and Antimicrobials in Cattle Manure Using Thermophilic Anaerobic Digestion (TAD)

The fate and effect of hormone and antimicrobial residues in the manure of therapeutically treated cattle is of considerable concern because of the adverse effects of environmental loading of these chemicals. This example demonstrates the feasibility of biodegradation of tylosin (TYL), chlortetracycline (CTC), sulfamethazine (SMZ) and megestrol (MEG) using thermophilic anaerobic digestion (TAD).

Specifically, quantitative methods using HPLC-DAD-MS/MS analysis were developed to monitor the hormone and antimicrobials, spiked into manure obtained from unmedicated cattle. These manure samples were incubated in active TAD with other controls for a 28-day period. Significant reductions in the concentrations of TYL, CTC, SMZ and MEG were demonstrated using TAD. Thus, TYL and CTC underwent essentially complete degradation, while both SMZ and MEG were 80% degraded at the end of the study.

In addition, somewhat surprisingly, the presence of antimicrobials had no negative effects on process stability and caused no significant reduction in total methane production. This process has demonstrated value in reducing the hormone and antimicrobial load to the environment, and allowing use of postdigested biosolids as a regenerated fertilizer.

### Chemicals and Materials

TYL (CAS number 1401-69-0, C46H77NO17, 916.10 g/mol, 8 mg/mL dissolved in aqueous 0.9% NaCl), CTC (CAS number 57-62-5, C22H23ClN2O8, 478.88 g/mol) and SMZ (CAS number 57-68-1, C12H14N4O2S, 278.33 g/mol), were purchased from Sigma-Aldrich (St. Louis, Mo., USA). MEG (CAS number 3562-63-8, C27H30O3, 342.47 g/mol) and formic acid were purchased from Fluka (St. Louis, Mo., USA). Microcrystalline cellulose was purchased From Acros (CAS number 9004-34-6, NJ, USA). Acetonitrile, citric acid, sodium hydroxide, ethylenediaminetetraacetic acid (EDTA) and methanol were purchased from Fisher Chemical (Fair Lawn, N.J., USA). 13 mm syringe filters (0.2 μm PTFE membrane) were purchased from VWR (Radnor, Pa. USA) and were used to filter samples prior to HPLC analysis. All water used in experiments was of Milli-Q quality.

Anaerobic digestate was freshly obtained from an active digester in an industrial biogas plant (Growing Power Hairy Hill, Vegreville, Alberta, Canada). The biogas plant operates three 10,400 m³ thermophilic anaerobic digesters with 2.5 MW of electricity generation capacity. Each digester is configured with a continuous stirred-tank reactor (CSTR) and the operating temperature is 55° C. Digesters receive up to 200 Mg of cattle manure and other biowaste per day, derived from an adjacent feedlot that produces 400 Mg of manure daily. The anaerobic digestate contains 8-10% total solids (TS) and 78.32±1.14% volatile solids (VS) out of the total solid content, with a pH of 7.6±0.13. Digestate was acclimatized in the lab digester for one week before it was used for experiments.

Control manure (free from the hormone and antimicrobials to be studied) was freshly collected from unmedicated dairy cattle at the Spring Creek Ranch (Vegreville, Alberta, Canada). Upon collection, the manure was stored at −20° C. The control manure was tested using the developed SPE-LC-MS/MS method and confirmed to contain no detectable amounts of TYL. CTC. SMZ or MEG.

### Extraction Method for TYL, CTC, SMZ and MEG

2.0 g of control manure were spiked with TYL, CTC, SMZ and MEG (1000 ppm stock solution, each prepared in 40:60 (v:v) acetonitrile/water) to prepare standards of 1.0, 10, 30, 50 and 100 ppm of each analyte. Samples were thoroughly mixed before extraction. The spiked manure was extracted twice with 10 mL of 8:2 (v/v) acetonitrile/0.25 M citric buffer (pH 4.0). After each extraction, the extracts were centrifuged (1400 RPM, 20 min): the supernatants were pooled and concentrated to remove the acetonitrile. 1 mL deionised water was then added to further dilute any remaining traces of acetonitrile. To prevent the chelating of antimicrobials standards with metals, 0.2 g EDTA was added to the extracts and vortexed for 30 seconds.

Solid phase extraction (SPE) was performed on a Grace Extract Clean™ C18 SPE cartridge (500 mg/8 mL) (Grace Davison Discovery Science, Deerfield, Ill., USA). The cartridge was preconditioned with 5 mL methanol followed by 5 mL deionised water. The supernatant of the extracts was then loaded into the cartridge, drained at a flow rate of 1 drop sec⁻¹ and cleaned with 5 ml, deionised water at 1 drop sec⁻¹. The sample was eluted from the cartridge using 10 mL of 9:1 (v/v) methanol/0.5 M citric buffer (pH 4.0). The resulting mixtures were then passed through a syringe filter prior to HPLC-DAD-MS/MS analysis. The recovery of the compounds was calculated using the peak area of the individual compound after extraction and the peak area of the compound with the same concentration that was dissolved in eluant.

### HPLC-DAD-MS/MS Analysis

Chromatographic separations were performed on a Thermo LCQ fleet ion trap HPLC-DAD-ESI-MS system (Thermo Scientific, San Jose, Calif., USA) consisting of an LC pump, an autosampler, a 6-port injection valve with a 20 μL injection loop, a DAD detector (diode array detector), an ESI (electrospray ionization) source and an ion trap mass spectrometry (MS) detector. The temperature of the column oven was set at 30° C. The temperature of the autosampler tray was set at 6° C. to prevent degradation of the hormone and antimicrobials. An Agilent Zorbox Eclipse PAH RPLC column (250×3.0 mm I.D., 5 μm) (Agilent, Santa Clara, Calif., USA) was used for the separation of the hormone and antimicrobials.

The separation of the one hormone (MEG) and three antimicrobials (TYL, CTC, SMZ) was achieved by an HPLC gradient method. Solvent A was acetonitrile and solvent B was 0.1% formic acid in H₂O. The gradient started with 20% A, increased to 55% within 5 min, to 95% within 1 min, was kept at 95% for 5 min, returned to initial composition within 2 ruin, and equilibrated within 3 min for a total run time of 15 min. The mobile phase flow rate was 500 μL/min. The analytes were detected in tandem by DAD and mass spectrometry. The DAD detection range was set from 250 to 400 nm. Individual MS tuning for each hormone and antimicrobial was done by infusing a 10 ppm standard with a flow rate of 10 μL/min into an individual mobile phase at 500 μL/min. Each mobile phase was chosen based on the composition when the individual standard was eluted. All the analytes were detected in positive ESI mode.

The resulting optimal ESI parameters for each compound are shown in Table S1. The Thermo LCQ fleet ion trap system was run in collision induced mode (MS2) for confirmative identification and increased sensitivity. The precursor and product ions used for TYL, CTC, SMZ and MEG identification and quantitation are listed in Table S2, along with associated collision energies.

TABLE S1
Optimal ESI parameters for the detection
of each hormone and antibiotic.

| parameter | S [symbol] | CTC | TYL | MEG |
|---|---|---|---|---|
| sheath gas (arb) | 20 | 20 | 20 | 30 |
| aux. gas (arb) | 10 | 10 | 20 | 0 |
| sweep gas (arb) | 0 | 0 | 0 | 0 |
| spray voltage (kV) | 5.0 | 5.0 | 5.0 | 5.0 |
| capillary temp. (° C.) | 270 | 270 | 270 | 270 |
| capillary voltage (V) | 10 | 31 | 41 | 10 |
| tube lens (V) | 64 | 59 | 79 | 64 |

[symbol]

indicates data missing or illegible when filed

TABLE S2
Tandem MS settings for each hormone and antibiotic.

| | precursor | product | collision |
|---|---|---|---|
| standard | ion [symbol] | ion [symbol] | energy [symbol] |
| S [symbol] | 279.1 | 124 [symbol] 186 | 30 |
| CTC | 479.1 | 462 [symbol] 444 | 35 |
| TYL | 916.3 | 772 [symbol] 2 | 23 |
| MEG | 385.1 | 267.2 [symbol] 325.1 | 30 |

[symbol]

indicates data missing or illegible when filed

### Anaerobic Digestion

The biodegradation experiment was set up in a lab-scale digester in which acclimatized thermophilic anaerobic digestate, as inoculum, and control manure slurry, with spiked hormone and antimicrobial standards, were mixed. The manure slurry was prepared by mixing 150 g of organic cattle manure with sterile water at 1:1 (w/v), and then spiked with TYL, CTC, SMZ and MEG standards to give 100 ppm of each compound in the stock mixture. 10 mL of the spiked manure slurry and 40 mL of digestate inoculum were added to each lab digester bottle (four replicates, bottle capacity 250 mL) for a final concentration of 20 ppm for each analyte (group 1). Digester bottles were sealed with butyl rubber septa and purged with pure nitrogen gas to resume anaerobic conditions. All digester bottles were placed in an incubator with a temperature setting of 55° C. and the anaerobic digestion process allowed to proceed.

Sampling and analysis was carried out at day 0, 2, 7, 14, 21 and 28 with a set of four digester bottles at each sampling interval. Control groups (with/without manure or TAD inoculum) were established to expose the effect of TAD on the degradation of the hormone and antimicrobials, Non-TAD/manure controls with different temperature settings were prepared. Thus, four replicate digester bottles, containing 40 mL of sterile water and 10 mL of the 100 ppm hormone and antimicrobials spiked in deionized water with a final concentration of 20 ppm for each analyte, were placed in an incubator at 55° C. (group 2). An analogous set of four replicate digester bottles were placed in an incubator at 22° C. (group 4). To test the effect of manure on the degradation of antimicrobials and hormone, four replicate digester bottles, containing 40 mL of sterile water and 10 mL of 100 ppm antimicrobials and hormone spiked in manure slurry with a final concentration of 20 ppm for each analyte, were placed in an incubator at 55° C. (group 3). To observe the effect of the hormone and antimicrobials on the TAD process, additional biogas production controls were set up, including TAD inoculum plus 10 mL of water: and TAD inoculum plus 10 mL water and 0.6 g crystalline cellulose (as a nutrient source for the microbes). The complete, detailed experimental design is shown in Table 4.

TABLE 4
Experimental design for biodegradation of hormones
and antimicrobials using TAD process

| content of digester | temp. | | TAD | manure | water |
|---|---|---|---|---|---|
| bottle (n − 4) | (° C.) | group # | (mL) | (mL) | (mL) |
| antimicrobials/hormone-manure + TAD | 55 | 1 | 40 | 10 | 0 |
| antimicrobials/hormone-manure + H₂O | 55 | 2 | 0 | 10 | 40 |
| antimicrobials/hormone + H₂O | 55 | 3 | 0 | 0 | 50 |
| antimicrobials/hormone + H₂O | 22 | 4 | 0 | 0 | 50 |

During the TAD process, biogas samples were taken from the headspace of the bottle at day 2, 7, 14, 21 and 28 from the TAD plus hormone/antimicrobials, TAD control and TAD plus cellulose control, respectively. After depressurization oldie digester bottles, 20 mL biogas was drawn using a syringe, injected into pre-vacuumed exetainers (Labco, Houston, Tex.) and analyzed by gas chromatography (Varian 450-GC) for methane and carbon dioxide composition.

Biogas production was calculated after normalization and expressed as accumulated biogas production during the period of study. For extraction of the samples containing TAD and spiked manure slurry (group 1 and group 2), 5 mL aliquots from each digester bottle at different sampling time intervals were transferred to a 50 mL centrifuge vial. Each aliquot was extracted twice using the developed method outlined previously. For the samples containing no TAD or manure slurry, 5 mL aliquots from each control bottle at different sampling time intervals were drawn and directly loaded into a C18 SPE cartridge at a flow rate of 1 drop sec⁻¹. The samples were eluted with 10 mL of 9:1 (v/v) methanol/0.5 M citric buffer (pH 4.0). All eluates were then passed through a syringe filter prior to HPLC-DAD-MS/MS analysis.

The biodegradation efficiency, as a function of time, of the TYL, CTC, SMZ and MEG standards was determined using the peak area of the day n product and the peak area of the day 0 product. Statistical analysis was performed on the effect of antimicrobials on methane production between TAD spiked with selected hormone and antimicrobial standards, and the TAD control.

The student's t-test was used at different sampling time intervals, and for comparison of cumulative methane production during the whole period of the experiment using the Chi-square test. The significance level was set at 95% (p<0.05).

### Results

Extraction Efficiency and Recovery of Compounds from Manure.

Extraction efficiencies and recovery of the selected hormone and antimicrobials from manure slurry using the extraction method are shown in Table 5. Excellent recoveries of TYL, CTC, SW, and MEG were achieved, which illustrated the applicability of the developed extraction method. A standard mixture of 1.0 ppm TYL, CTC, SMZ and MEG are shown in a well resolved HPLC chromatograms (data not shown).

TABLE 5
Recovery of 1.0 ppm hormone and antimicrobial
standards spiked in organic cow manure

| | Recovers (%) |
|---|---|
| Standard | (n = 3) |
| TYL | 96.7 ± 2.5 |
| CTC | 90.0 ± 11.3 |
| SMZ | 83.7 ± 8.4 |
| MEG | 81.4 ± 2.4 |

Calibration curves of selected antimicrobial and hormone standards were generated by subjecting the analytes to the developed extraction process. Four concentrations of the hormone and antimicrobial standards at 1.0, 10, 30, 50 and 100 ppm respectively were spiked into 2.0 g of control manure slurry, extracted, and subsequently analyzed by HPLC-DAD-MS/MS. Linear calibration curves were obtained for all standards up to the 100 ppm range (R2-0.991, 0.991 and 0.993 for TYL SMZ and MEG, respectively), with the exception of CTC, which performed a linear response up to 50 ppm (R2=0.996).

### Fate of TYL, CTC, SMZ and MEG in TAD and Different Controls

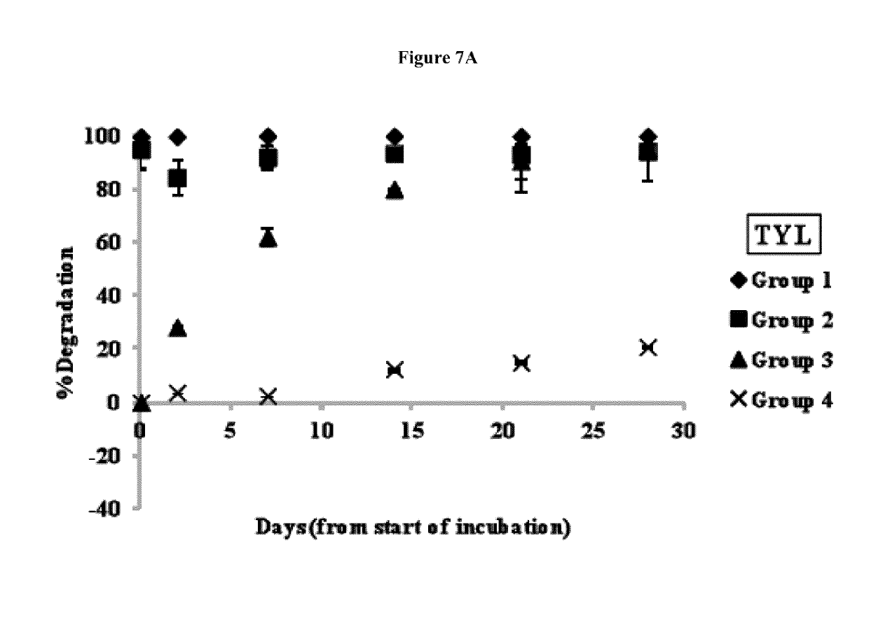

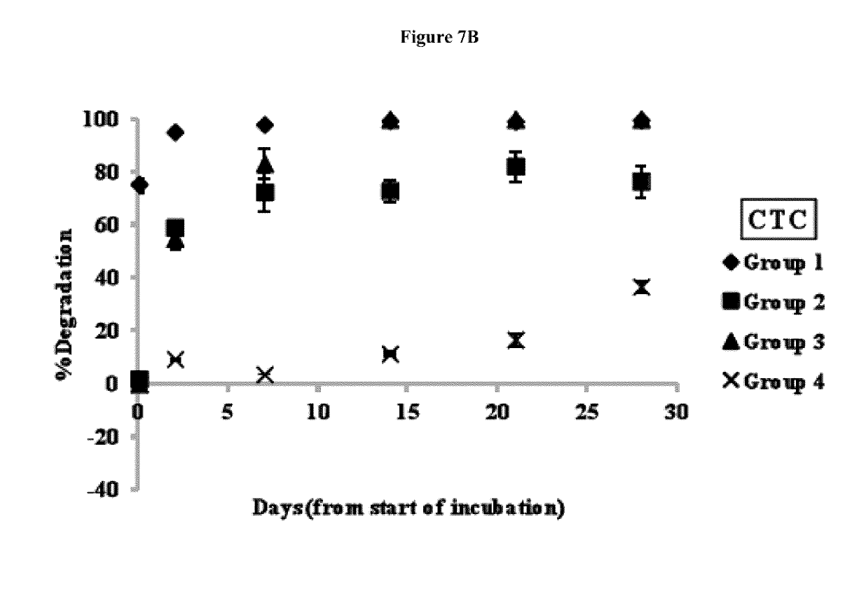

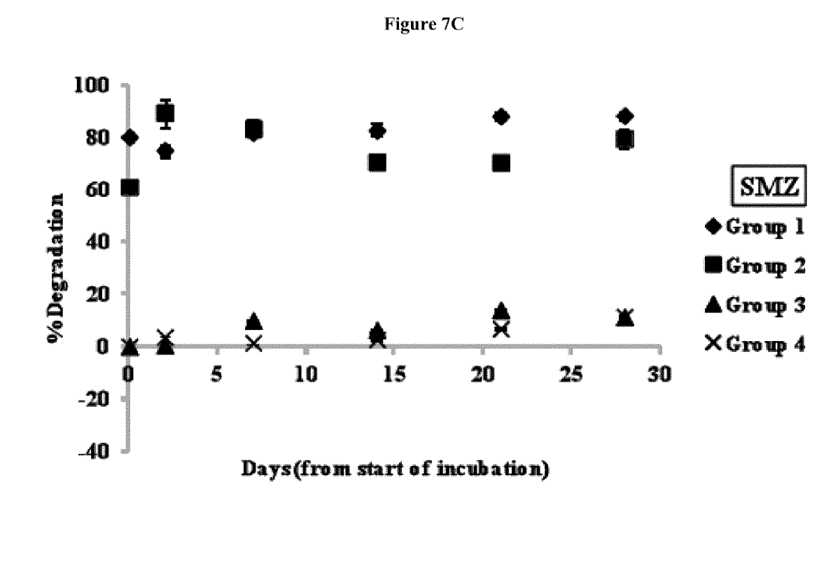

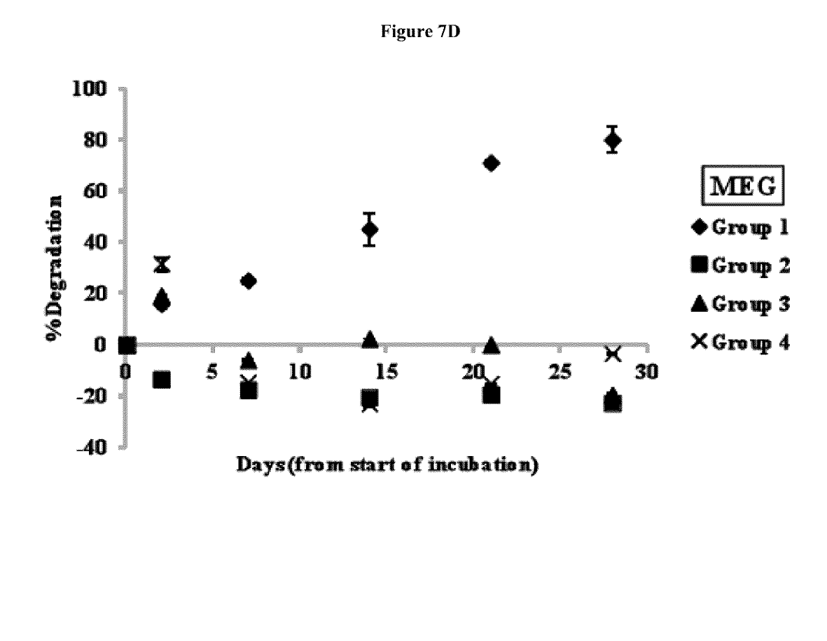

Different levels of biodegradation of the spiked hormone and antimicrobials were observed in this study (FIGS. 7A-7D). To calculate the percentage of biodegradation in different experimental settings at day 0, the MS peak area of the hormone and antimicrobial standards spiked in the water control at 22° C. was used as the baseline. In the case of groups 1 and 2, significant degradation of the parent antimicrobials (TYL, CTC and, for group 1 only, SMZ) appeared to occur during day zero.

The degradation profiles of the selected hormone and antimicrobials at different experimental settings demonstrated clearly that both TYL, and CTC have significant thermal lability. During the 28-day study period, TYL and CTC underwent less than 40% degradation at ambient temperature (22° C.) while at least 80% degradation was observed at 55° C. with or without the TAD/manure component. Complete CTC degradation was observed by day 14 and 90% TYL degradation by day 28 in the water control group at 55° C. By contrast, SMZ and MEG showed much greater thermal stability. There was no significant degradation (e.g., <20%) observed, nor variation in degradation between ambient and elevated temperatures. During the 28-day study period, SMZ was degraded less than 20% in the water control at both 55 and 22° C. No observed degradation of MEG occurred in the water control at either 55 or 22° C., or in the manure plus water control at 55° C.

Interestingly, the degradation profile of SMZ in the water plus manure control demonstrates the microbial effect on selected antimicrobials in comparison with the water control at 55 CC. SMZ underwent little appreciable degradation in the absence of manure, but reached almost 80% degradation in the presence of manure by the end of the study. The manure contains a consortium of microbes similar to the well-established ones found in active anaerobic digestion. This was evident in the biogas production associated with that group. Methane production commenced after incubation in anaerobic conditions at 55° C. for 21 days (1.028 Nml/g VS, average) and increased to 6.872 Nml/g VS at day 28 because the initial mass of manure (˜1.3 g) with microbes was small in each digester bottle and it took time to acclimatize and propagate for methane production.

Over 80% degradation of TYL was achieved almost immediately in the presence of manure and elevated temperatures while the same level of degradation was seen in water without manure only on day 21. The degradation profile for CTC in the manure plus water control group at 55° C. appeared anomalous in comparison with the active TAD group, and the water without manure at elevated temperature.

Of note, the best degradation profiles for selected hormone and antimicrobials were observed in the well-established and active thermophilic anaerobic digestion group. TYL and CTC underwent essentially complete degradation by day 7 of the TAD process. This represents a moderate increase in both rate and extent of degradation relative to the manure plus water and water only groups at the same temperature. Additionally, there was a small increase in the extent of degradation of SMZ relative to the manure plus water group. However, degradation of SMZ in both groups showed a similar pattern and reached only 80% of degradation during this 28-day study. This indicated that TAD could destroy up to 80% of the parent form of SMZ a day or two following establishment of a healthy digestion process. The most noteworthy was the behavior of MEG and its dynamic degradation under TAD. This synthetic hormone was completely stable under all other conditions in this study, exhibiting no degradation at elevated temperatures or in the presence of limited amounts of microbes. However, under active and well-established TAD, almost 80% of the original dose of MEG was degraded during a 28-day digestion process, with a trend towards a complete degradation if the batch TAD digestion had been extended. This is particularly important given the environmental persistence of MEG and its structural analogues, and clearly illustrates the capability of TAD for the degradation of MEG.

Thus, TAD has been shown to be effective at degrading the selected hormone and antimicrobials. Elevated temperature and anaerobic microorganisms acted synergistically to achieve the best degradation, particularly for the hormone MEG. Though the mechanism of degradation of hormones and antimicrobials is not completely known, it is anticipated that the synergistic effect of microbial activities, high pH, elevated temperature and presence of other small molecules will destabilize the selected hormone and antimicrobials leading to effective degradation.

### Effect of TYL, CTC, SMZ and MEG on Biogas Production

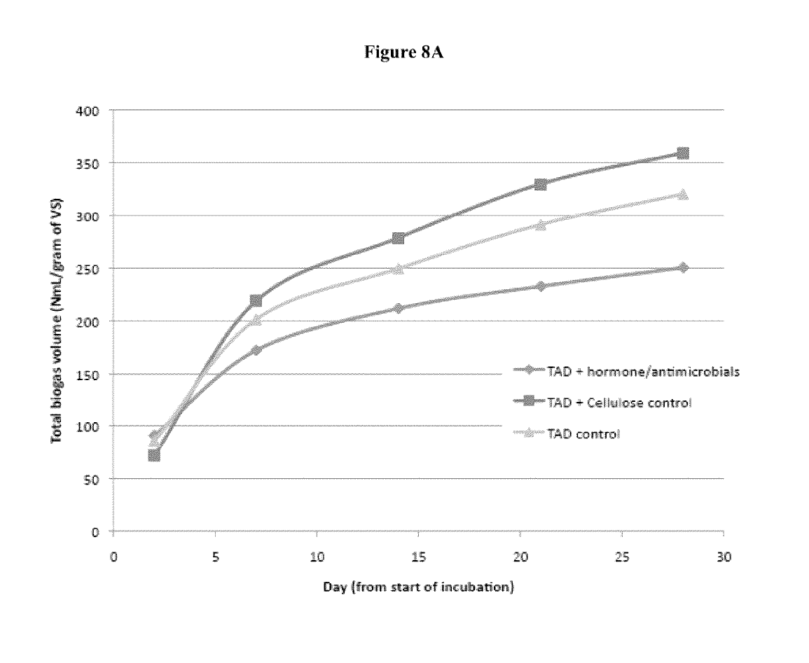

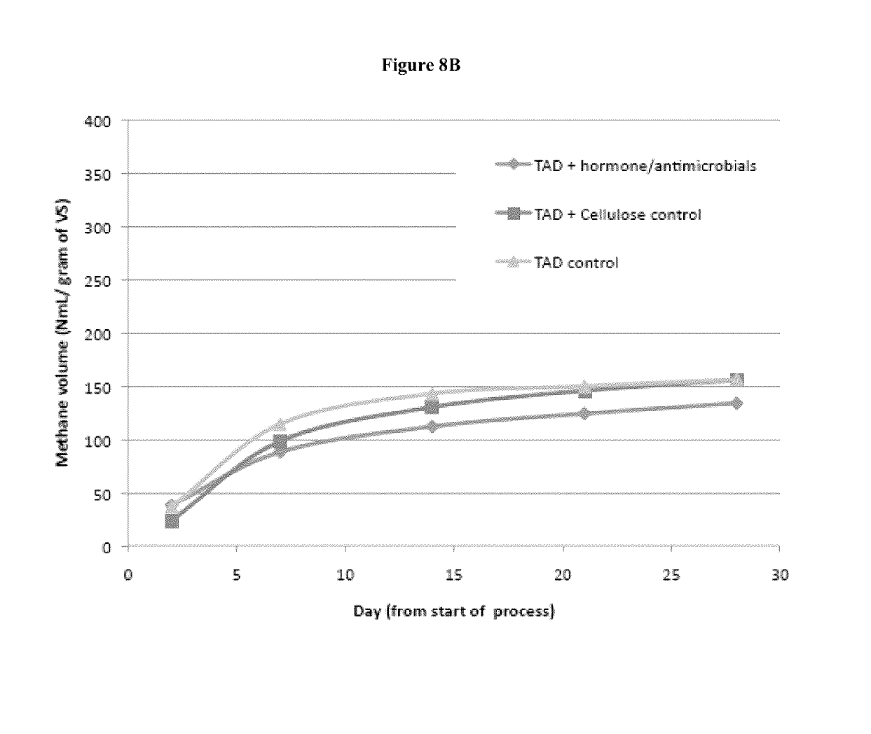

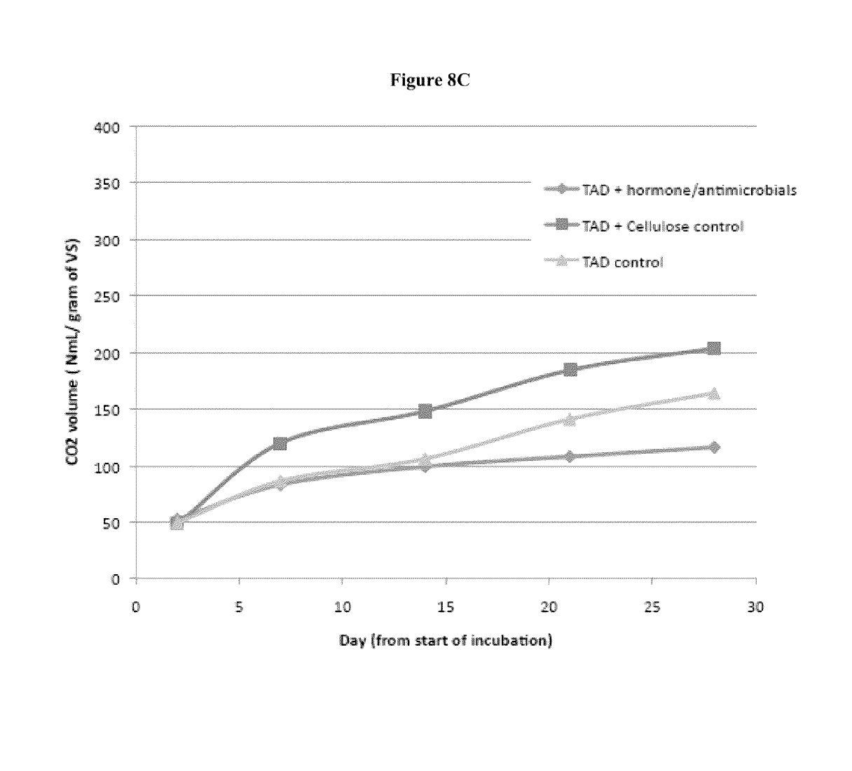

Batch assays were incubated at 55° C. for 28 days. Cumulative biogas production in each assay was shown in FIGS. 8A-8C.

Biogas production was measured during the study period for the following: a TAD control; a TAD plus cellulose control TAD plus the hormone and antimicrobials. There was no obvious lag phase in biogas production in TAD containing the hormone/antimicrobials. However, methane production was lower in the TAD plus hormone/antimicrobials group than that in the TAD and TAD plus cellulose controls between day 3 and 7 (50.139±3.589 versus 77.916±5.278 and 75.023±31.991 normalized ml (Nml)/g volatile solid (g VS), p=0.0001, t-test), and between day 8 and 14 (23.893±3.968 versus 28.594±3.589 2.007 and 31.904±1.233 Nml/g VS, p<0.004, t-test). Inhibition of methane production of TAD at the current doses of 20 ppm each of CTC, TYL and SMZ in the mixture was observed between day 3 and 7 (35.6%) and day 8 and 14 (16.4%) in this study. There was no significant reduction of methane production in TAD containing CTC, TYL and SMZ observed from day 15 to 21 and day 22 to 28 (p=0.82 and 0.31). For the entire period of the study, total cumulative methane production was 156.61 and 134.18 NmL/g VS for the TAD control and TAD containing the hormone and antimicrobials.

There was no statistically significant difference between the two groups in terms of total methane production (p=0.06, chi-square test). In fact, increased cumulative methane production was observed during day 15 to 28 for TAD with the hormone and antimicrobials (21.77 Nml/gVS) versus the TAD control (13.34 Nml/g VS). This could explain why there is no significant impact on total methane production.

Meanwhile, the carbon dioxide productivity was also inhibited in TAD with the hormone and antimicrobials (cumulative CO2 116.2 NmL/g VS), compared to the TAD control (164.1 NmL/g VS). These results demonstrated that the three antimicrobials under study did not only inhibit methanogens, but also slowed down catalytic and metabolic activities of the entire consortium of microorganisms that exist in an active TAD process. The combined effects of TYL, CTC, and SMZ at 20 ppm level caused only a moderate reduction of total biogas production during the 28-day period.

Previous studies have reported varied level of inhibition of antimicrobials on biogas production during anaerobic digestion of pig and cow manure, in the presence of CTC and oxytetracycline (OTC). However, a broad disagreement is observed in the literature regarding the extent of the inhibitory effect of CTC and OTC. At an extreme, Lallai et al. (Bioresour. Technol. 2002, 82 (2), 205-208) observed essentially no inhibitory effect with OTC concentrations as high as 250 ppm, whilst Arikan of al. (Process Biochem, 2006, 41 (7), 1637-1643) observed a 27% reduction in methane production with the lowest dose of 3.1 ppm OTC. The majority of data, however, suggests inhibition of methane production in the presence of CTC and OTC, with the effect of CTC tending to be more pronounced.

With no or at best a modest inhibition of biogas production in current study, the energy and economic value of the biogas is not significantly compromised. Under the concept of a fully integrated utilization of biowaste to energy, additional value of post-digested biosolids as a regenerated organic fertilizer could be realized if hormones and antimicrobials are readily degraded in a biological and environmentally friendly way.

### REFERENCES

(1) Sarmah et al., Chemosphere 2006, 65 (5). 725-759.
(2) Zheng et al., Environ. Sci. Technol. 2008, 42 (2), 530-535.
(3) Animal Health Institute Website (ahi dot org).
(4) Jjemba, Agric. Ecosyst. Environ. 2002, 93 (1-3), 267-278.
(5) Kumar et al., Adv. Agron. 2005, 87, 1-54.
(6) Hanselman et al., Environ. Sci. Technol. 2003, 37 (24), 5471-5478.
(7) Boxall et al., Environ. Sci. Technol. 2003, 37 (15), 286A-294A.
(8) United States Department of Agriculture: National Agricultural Statistics Service Website (nass dot usda dot gov).
(9) Arikan et al., Bioresour. Technol. 2009, 100(19), 4447-4453.
(10) Arikan et al., Process Biochem. 2006, 41 (7), 1637-1643.
(11) Bao et al., Waste Manage. 2009, 29(4), 1416-1423.
(12) Dolliver et al., J. Environ. Qual. 2008, 37 (3), 1245-1253.
(13) Alvarez et al., Bioresour. Technol. 2010, 101 (22), 8581-8586.
(14) Lindsey et al., Anal. Chem. 2001, 73 (19), 4640-4646.
(15) Chee-Sanford et al., Appl. Environ. Microbiol. 2001, 67 (4), 1494-1502.
(16) Kolodziej et al., Environ. Sci. Technol. 2004, 38 (23), 6377-6384.
(17) Yamamoto et al., Environ. Sci. Technol. 2006, 40 (13), 4132-4137.
(18) Khanal et al., Environ. Sci. Technol. 2006, 40 (21), 6537-6546.
(19) Arikan et al., Desalination 2008, 226 (1-3), 121-133.
(20) Ermawati et al., J. Biosci. Bioeng. 2007, 103 (1), 27-31.
(21) Murray et al., Eds. Manual of Clinical Microbiology, 9th, ed.: American Society for Microbiology: Washington, 2007; pp 787.
(22) Smith et al., Waste Manage. 2005, 25 (4), 417-425.
(23) Wagner et al., Anaerobe 2008, 14 (3), 181.183.
(24) Gao et al., Bioresour. Technol. 2012, 110, 692-696.
(25) Scanlon et al., Can. J. Anim Sci. 1972, 52, 767-769.
(26) Schiffer et al., Sci. Total Environ. 2004, 326 (1-3), 225-37.
(27) Liu et al., Sci. Total Environ. 2011, 409 (24), 5149-61.
(28) Lanai et al., Bioresour. Technol. 2002, 82 (2), 205-208.
(29) Sanz et al., Appl. Microbiol. Biotechnol. 1996, 46 (5-6), 587-592.
(30) Loftin et al., Environ. Toxicol, Chem. 2005, 24 (4), 782-788.

All references and publications cited herein are incorporated by reference.

1. A method for accelerating and/or enhancing degradation of an antibiotic that may be present in a carrier material (such as an organic waste), comprising providing the carrier material to an anaerobic digestion (AD) reactor, and optionally maintaining the rate of biogas production substantially steady during the AD process.

2. The method of claim 1, wherein the antibiotic does not substantially degrade by high temperature alone (e.g., incubation at about 50-60° C. or 55° C.).

3. The method of claim 2, wherein the antibiotic is a sulfonamide antibacterial agent that includes a sulfonamide group (e.g., sulfamethazine).

4. The method of claim 1, wherein the antibiotic is a veterinary medicine.

5. The method of claim 1, wherein the antibiotic is present in a biowaste material (e.g., animal meat, animal manure, or other animal body fluid or excretes) or some by-products (e.g., WDGS).

6. The method of any of claims 1-5, wherein the AD reactor is operated in batch mode, semi-continuous mode, or continuous mode.

7. The method of claim 6, wherein the batch mode lasts less than about 0.5 hr, 1 hr, 2 hr, 5 hr, 10 hr, 24 hr, 2, 3, 4, 5, 6, 7, 10, 20, 30, 40, 50, or 60 days.

8. The method of claim 6 or 7, wherein the rate of biogas production peaks at about 0.5-5 hrs, 1-7 days, or 5-10 days after the beginning of the batch mode operation.

9. The method of any of claims 1-8, wherein a carbon-rich material is provided, semi-continuously to the AD reactor once every about 0.5-5 hrs, 1-7 days, or 5-10 days after reaching peak biogas production, to maintain substantially steady biogas production.

10. The method of any of claims 1-9, wherein the AD reactor contains an active inoculum of microorganisms at the beginning of the batch mode operation.

11. The method of any of claims 1-10, wherein the AD process is carried out by a consortium of anaerobic microorganisms, such as psyclophilic microorganisms, mesophilic microorganisms, or thermophilic microorganisms.

12. The method of claim 11, wherein the thermophilic microorganisms are acclimatized with substrates containing proteins with abundant β-sheets.

13. The method of claim 12, wherein the thermophilic microorganisms are acclimatized by culturing with substrates containing amyloid substance at elevated temperature and extreme alkaline pH.

14. The method of any of claims 1-13, further comprising adding one or more supplemental nutrients selected from Ca, Fe, Ni, or Co.

15. The method of any of claims 1-14, wherein the AD is carried out at about 20° C., 25° C., 30° C., 37° C., 40° C., 45° C., 50° C., 55° C., 60° C., or above.

16. The method of any of claims 1-15, wherein >50%, 60%, 70%, 80%, 90% or more (in terms of amount) of the antibiotic is degraded after about 2 days to 2 weeks of anaerobic digestion.

17. The method of claim 1, wherein the carrier material is a biowaste material (e.g., animal meat, animal manure, or other animal body fluid or excretes) or WDGS.

18. The method of claim 1, wherein more than 50%, 60%, 70%, 80%, 85%, 90%, 95% 99% or nearly 100% of the antibiotic in the carrier material is degraded.

19. The method of claim 1, wherein the rate of biogas production is reduced by 110 more than 40%, 30%, 25%, 20%, 15%, 10%, 5% or less; or the rate of methane production is reduced by no more than 25%, 20%, 15%, 10%, 5% or less.

20. The method of claim 1, wherein the antibiotic is present in the AD reactor at a concentration no more than 100 ppm, 80 ppm 60 ppm, 40 ppm, 20 ppm, 10 ppm, 5 ppm, 3 ppm, 2 ppm, 1 ppm or less.
